(19) United States
(12) Patent Application Publication
(10) Pub. No.: US 2025/0268582 A1
(43) Pub. Date: Aug. 28, 2025

(54) SYSTEM, METHOD AND DEVICE FOR REMOTE ULTRASONOGRAPHY

(71) Applicant: Wosler Corp., Calgary (CA)

(72) Inventors: Marco Brandon Steven GALLONE, Calgary (CA); Leah Anne GROVES, Kingston (CA); Matthew Peter Cary DA SILVA, London (CA); Ankur Jai SOOD, Oakville (CA); Yue ZHOU, London (CA); Jasper Johannus Willem DE REGT, Denfield (CA); Henry Ifeanyi MADUBUOBI, Calgary (CA)

(21) Appl. No.: 18/858,101

(22) PCT Filed: Apr. 18, 2023
(86) PCT No.: PCT/CA2023/050520

§ 371 Date: Oct. 18, 2024

Related U.S. Application Data
(60) Provisional application No. 63/332,498, filed on Apr. 19, 2022.

(51) Int. Cl. A61B 8/00

(52) U.S. Cl. CPC A61B 8/54; A61B 8/4218; A61B 8/4254; A61B 8/467; A61B 8/56

(57) ABSTRACT

The present disclosure generally relates to systems, methods and devices for remote ultrasonography. The system may include a local control system comprising an input device and at least one local processor coupled to the input device; a remote robotic ultrasound system comprising a motion subsystem and at least one local processor coupled to the motion subsystem, wherein the at least one local processor, of the local control system, is operable to: detect a new positional state for the input device; obtain positional data corresponding to the new positional state; generate, based on the positional data, input-side motion demand data; and transmit the input-side motion demand data to the remote robot system, wherein the at least one remote processor, of the remote robot system, is operable to: receive the input-side motion demand data; and adjust a positional state of the motion subsystem, based on the input-side motion demand data.

### FIELD

The present disclosure relates generally to ultrasonography, and in particular, to systems, methods and devices for remote ultrasonography.

### INTRODUCTION

Medical imaging tools are often used for evaluating, assessing and diagnosing medical conditions. It is estimated that approximately two thirds of medical conditions are assessable through medical imaging techniques. Among the various available imaging options, ultrasound imaging is widely considered to be a safe, cost-effective and pragmatic option.

However, ultrasound imaging typically requires the availability of highly trained, and highly skilled technicians (e.g., sonographers) to operate complex ultrasound equipment. This presents challenges in providing sonographic services to many remotely situated and under-serviced communities. In particular, patients in these communities are often forced to travel long distances to reach the nearest urban center where ultrasound scanning services are readily available.

### SUMMARY

The following introduction is provided to introduce the reader to the more detailed discussion to follow. The introduction is not intended to limit or define any claimed or as yet unclaimed invention. One or more inventions may reside in any combination or sub-combination of the elements or process steps disclosed in any part of this document including its claims and figures.

In one broad aspect, there is provided system for remote ultrasonography, comprising: a local control system comprising an input device and at least one local processor coupled to the input device; a remote robotic ultrasound system comprising a motion subsystem and at least one local processor coupled to the motion subsystem, wherein the at least one local processor, of the local control system, is operable to: detect a new positional state for the input device; obtain positional data corresponding to the new positional state; generate, based on the positional data, input-side motion demand data; and transmit the input-side motion demand data to the remote robot system, wherein the at least one remote processor, of the remote robot system, is operable to: receive the input-side motion demand data; and adjust a positional state of the motion subsystem, based on the input-side motion demand data.

The at least one remote processor, of the remote robotic ultrasound system, may be further operable to: monitor for changes in the positional state of the motion subsystem; and in response to detecting a change in the positional state, generate robot-side motion demand data corresponding to a new position state of the motion subsystem; and transmit the robot-side motion demand data to the local control system.

The at least one local processor, of the local control system, may be further operable to: receive the robot-side motion demand data; and adjust a positional state of the input device, based on the robot-side motion demand data, so as to generate a haptic feedback effect at the input device.

The input device can include a sensor subsystem, and detecting a new positional state for the input device can include receiving new sensor data from the sensor subsystem.

The sensor subsystem can include a plurality of rotary encoders, and the positional data can include encoder data generated by the plurality of rotary encoders.

The sensor subsystem can include an inertial measurement unit (IMU), and the positional data can include IMU data generated by the IMU.

Generating the input-side motion demand data can include determining an input-side transformation matrix, based on the positional data, where determining the input-side transformation matrix includes solving a forward kinematics model.

Adjusting a positional state of the motion subsystem can include analyzing the input-side transformation matrix by solving an inverse kinematics model.

Generating robot-side motion demand data can include determining a robot side transformation matrix, based on the new positional state, where determining the robot-side includes solving a forward kinematics model.

The input device can include a movable arm system.

The motion subsystem can include a robotic arm.

The motion subsystem can include a tool-retaining end effector for retaining a tool.

The end effector can include a first connector which engages with a second connector associated with the tool.

The tool can include one or more of an ultrasound transducer, gel dispenser and a transvaginal probe.

The tool can be disposed inside of a casing that includes a flexible sock, and the second connector can be attached to the casing.

The input-side motion demand data can control one of a spatial and an orientation position of the tool-retaining end effector.

The local control system can include an input interface, and at least one local processor of the local control system can be operable to: receive one or more user inputs from the input interface; and transmit the one or more user inputs to the remote robotic ultrasound system.

The one or more user inputs can include one of an ultrasound transducer configuration and a command to exchange an actively-selected tool.

The input interface can include an ultrasound input interface.

The remote robotic ultrasound system can include a pain threshold monitor coupled to the at least one remote processor of the remote robotic ultrasound system, and where the at least one remote processor of the remote robotic ultrasound system is further operable to: monitor for an activation signal from the pain threshold monitor; and in response to detecting the activation signal, one of disabling the motion subsystem and reducing an applied force of the motion subsystem on a patient.

In another broad aspect, there is provided a method for remote ultrasonography, comprising: detecting a new positional state for an input device of a local control system; obtaining positional data corresponding to the new positional state; generating, based on the positional data, input-side motion demand data; and transmitting, from the local control system, the input-side motion demand data to a remote robot system, receiving, at the remote robot system, the input-side motion demand data; and adjusting a positional state of a motion subsystem, of the remote robot system, based on the input-side motion demand data.

The method can include: monitoring, at the remote robot system, for changes in the positional state of the motion subsystem; and in response to detecting a change in the positional state, generating robot-side motion demand data corresponding to a new position state of the motion subsystem; and transmitting the robot-side motion demand data to the local control system.

The method can include: receiving, at the local control system, the robot-side motion demand data; and adjusting, at the local control system, a positional state of the input device, based on the robot-side motion demand data, so as to generate a haptic feedback effect at the input device.

The input device can include a sensor subsystem, and detecting a new positional state for the input device can include receiving new sensor data from the sensor subsystem.

The sensor subsystem can include a plurality of rotary encoders, and the positional data can include encoder data generated by the plurality of rotary encoders.

The sensor subsystem can include an inertial measurement unit (IMU), and the positional data can include IMU data generated by the IMU.

Generating the input-side motion demand data can include determining an input-side transformation matrix, based on the positional data, where determining the input-side transformation matrix includes solving a forward kinematics model.

Adjusting a positional state of the motion subsystem can include analyzing the input-side transformation matrix by solving an inverse kinematics model.

Generating robot-side motion demand data can include determining a robot side transformation matrix, based on the new positional state, where determining the robot-side includes solving a forward kinematics model.

The input device can include a movable arm system.

The motion subsystem can include a robotic arm.

The motion subsystem can include a tool-retaining end effector for retaining a tool.

The end effector can include a first connector which engages with a second connector associated with the tool.

The tool can include one or more of an ultrasound transducer, gel dispenser and a transvaginal probe.

The tool can be disposed inside of a casing that includes a flexible sock, and the second connector can be attached to the casing.

The input-side motion demand data can control one of a spatial and an orientation position of the tool-retaining end effector.

The local control system can include an input interface, and the method can include: receiving, at the local control system, one or more user inputs from the input interface; and transmitting, from the local control system, the one or more user inputs to the remote robotic ultrasound system.

The one or more user inputs can include one of an ultrasound transducer configuration and a command to exchange an actively-selected tool.

The input interface can include an ultrasound input interface.

The remote robotic ultrasound system can include a pain threshold monitor coupled to the at least one remote processor of the remote robotic ultrasound system, and the method can include: monitoring for an activation signal from the pain threshold monitor; and in response to detecting the activation signal, one of disabling the motion subsystem and reducing an applied force of the motion subsystem on a patient.

In accordance with another broad aspect, there is provided an input device comprising: a mounting structure; a user-controllable arm system, comprising: (i) a mechanical arm extending between a first arm end and a distal second arm end, and (ii) a rotatable member rotatably coupled to the mounting structure, wherein the mechanical arm is secured to the rotatable member at the second arm end; a motor subsystem comprising a plurality of motors for controlling a positional state of the arm system; a sensor subsystem for monitoring the positional state of the arm system; at least one processor coupled to each of the motor subsystem and the sensor subsystem.

The mechanical arm can include a plurality of arm segments in rotatable connection.

A user-engageable joystick can be mounted to the first arm end, the joystick can provide an engagement interface for moving the arm system.

The joystick can include one or more contact sensors coupled to the at least one processor.

The joystick can include an activation toggle coupled to the at least one processor.

The input device can include a power supply coupled to the at least one processor, where the at least one processor is operable to control power supplied to the motor subsystem from the power supply.

When the contact sensors are not engaged, the at least one processor can supply a current, from the power supply, to the motor subsystem to hold the arm system in a static position.

The at least one processor can be operable to: receive motion demand data from a remote system; and apply increased current, to one or more motors of the motor subsystem, to increase motor torque and generate a haptic feedback effect.

The rotatable connections between the arm segments, as well as between the rotatable member and the mounting structure, can include rotatable shafts.

The sensor subsystem can include one or more rotary encoders coupled to the rotatable shafts, each rotary encoder generating encoder data corresponding to a rotational position of a respective shaft.

One or more of the rotatable shafts can be coupled to a respective motor of the motor subsystem.

The coupling can be one of a direct coupling and an indirect coupling, and the indirect coupling can include a belt and pulley mechanism.

The at least one processor can be operable to control the motor subsystem to adjust a rotational position of a rotational shaft in order to adjust the positional state of the arm system.

The sensor subsystem can include an inertial measurement unit mounted to the joystick.

The user-controllable arm system can include, (i) an orientational-position control portion; and (ii) a spatial-position control portion, where the orientational-position control portion is configured to control an orientational position of an end effector associated with a motion subsystem in a remote environment, and the spatial-position control portion is configured to vary a spatial position of the end effector.

The orientational control portion can include at least one arm segment of the mechanical arm.

The at least one arm segment can include a forward wrist arm segment, a mid-arm rotation segment and a rear arm rotation segment.

The forward wrist arm segment can be rotatably coupled to the mid-arm rotation segment, and rotate about a first yaw axis to control a yaw angle of the end effector in the remote environment.

The mid-arm rotation segment can be rotatably connected to the rear arm rotation segment, and rotate about a second pitch axis to control a pitch angle of the end effector in the remote environment, where the second axis is orthogonal to the first axis.

The spatial-position control portion can include one or more arm segments of the mechanical arm, as well as the rotatable member.

The one or more arm segments can include a forward translation arm segment and a rear translation arm segment.

The rear arm rotation segment can be rotatably connected to the forward translation rotation segment, and rotate about a third roll axis to control a roll angle of the end effector in the remote environment, where the third axis is orthogonal to both the first and second axis.

The forward translation arm segment can be rotatably connected to the rear translation arm segment, and rotate about a fourth axis to control vertical spatial motion of the end effector in the remote environment.

The rear translation arm segment can be rotatably connected to the rotatable member, and rotate about a fifth axis to control forward and backward spatial motion of the end effector in the remote environment.

The rotatable member can be rotatably connected to the mounting structure, and rotate about a sixth axis to control lateral horizontal spatial motion of the end effector in the remote environment.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

The drawings included herewith are for illustrating various examples of systems, methods, and devices of the present specification and are not intended to limit the scope of what is taught in any way. In the drawings:

FIG. 1A is a simplified block diagram of an example system for remote ultrasonography;

FIG. 1B is a schematic illustration of an example local control system in communication with a remote robotic ultrasound system;

FIG. 2 is a simplified electrical hardware block diagram of an example local control system in communication with a remote robotic ultrasound system;

FIG. 3 is a simplified block diagram illustrating data flow between a local control system and a remote robotic ultrasound system;

FIG. 4A is a side-front perspective view of an example input device associated with a local control system;

FIG. 4B is a side-rear perspective view of the input device;

FIG. 4C is a side elevation view of the input device;

FIG. 4D is a side cross-sectional elevation view of the input device, taken along the section line 4D-4D′ of FIG. 4A;

FIG. 4E is a side perspective cross-sectional view of the input device, taken along the section line 4D-4D′ of FIG. 4A;

FIG. 4F is a front perspective view of the input device;

FIG. 4G is a perspective cross-sectional view of the input device, taken along the section line 4F-4F′ of FIG. 4D;

FIG. 4H is a perspective cross-sectional view of the input device, taken along the section line 4H-4H′ of FIG. 4F;

FIG. 4I is a simplified electrical hardware block diagram of an example input device;

FIG. 5A is a schematic illustration of a tool, in a remote environment, being rotated in the clockwise and counter clockwise direction;

FIG. 5B is a schematic illustration of a tool, in a remote environment, being rolled forwards and backwards;

FIG. 5C is a schematic illustration of a tool, in a remote environment, being rolled leftward and rightward;

FIG. 5D is a schematic illustration of a tool, in a remote environment, being translated to different spatial positions;

FIG. 6A is a schematic illustration showing a side view of an example remote robotic ultrasound system;

FIG. 6B is a schematic illustration showing another side view of another example of the remote robotic ultrasound system;

FIG. 6C is a schematic illustration showing a front view of the remote robotic ultrasound system in FIG. 6B;

FIG. 7A is a schematic illustration showing a side view of an example robotic arm used in a remote robotic ultrasound system;

FIG. 7B is a schematic illustration showing a side view of another example robotic arm used in a remote robotic ultrasound system;

FIG. 8A is a simplified electrical hardware block diagram of an example end effector of a robotic arm;

FIG. 8B is an illustration of an example of a tool retention mechanism used with an end effector;

FIG. 8C is an illustration of another example tool retention mechanism used with an end effector;

FIG. 8D is an illustration of a number of tools disposed in a tool holder area;

FIG. 8E is an illustration of an example casing body for retaining an ultrasound transducer;

FIG. 8F is an illustration of an example casing body for retaining a transvaginal probe;

FIG. 8G is an illustration of another example tool retention mechanism used with an end effector;

FIG. 8H is an illustration of another example tool retention mechanism used with an end effector;

FIG. 9 is an illustration of an example setup configuration for various object tracking sensors and cameras within a remote environment;

FIG. 10 is a process flow for an example method for positional control and feedback between an input device and a remote robot system; and

FIG. 11 is a process flow for an example method for remote ultrasonography.

Further aspects and features of the examples described herein will appear from the following description taken together with the accompanying drawings.

### DETAILED DESCRIPTION

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

It should also be noted that the terms “coupled” or “coupling” as used herein can have several different meanings depending in the context in which these terms are used. For example, the terms coupled or coupling can have a mechanical, fluidic or electrical connotation. For example, as used herein, the terms coupled or coupling can indicate that two elements or devices can be directly connected to one another or connected to one another through one or more intermediate elements or devices via an electrical or magnetic signal, electrical connection, an electrical element or a mechanical element depending on the particular context. Furthermore coupled electrical elements may send and/or receive data.

Unless the context requires otherwise, throughout the specification and claims which follow, the word “comprise” and variations thereof, such as, “comprises” and “comprising” are to be construed in an open, inclusive sense, that is, as “including, but not limited to”.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Similarly, throughout this specification and the appended claims the term “communicative” as in “communicative pathway,” “communicative coupling,” and in variants such as “communicatively coupled,” is generally used to refer to any engineered arrangement for transferring and/or exchanging information. Exemplary communicative pathways include, but are not limited to, electrically conductive pathways (e.g., electrically conductive wires, electrically conductive traces), magnetic pathways (e.g., magnetic media), optical pathways (e.g., optical fiber), electromagnetically radiative pathways (e.g., radio waves), or any combination thereof. Exemplary communicative couplings include, but are not limited to, electrical couplings, magnetic couplings, optical couplings, radio couplings, or any combination thereof.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

The example systems and methods described herein may be implemented as a combination of hardware or software. In some cases, the examples described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g. a keyboard, mouse, touchscreen, or the like), and at least one output device (e.g. a display screen, a printer, a wireless radio, or the like) depending on the nature of the device.

Some elements that are used to implement at least part of the systems, methods, and devices described herein may be implemented via software that is written in a high-level procedural language such as object-oriented programming. The program code may be written in C++, C#, JavaScript, Python, or any other suitable programming language and may comprise modules or classes, as is known to those skilled in object-oriented programming. Alternatively, or in addition thereto, some of these elements implemented via software may be written in assembly language, machine language, or firmware as needed. In either case, the language may be a compiled or interpreted language.

At least some of these software programs may be stored on a computer readable medium such as, but not limited to, a ROM, a magnetic disk, an optical disc, a USB key, and the like that is readable by a device having at least one processor, an operating system, and the associated hardware and software that is used to implement the functionality of at least one of the methods described herein. The software program code, when read by the device, configures the device to operate in a new, specific, and predefined manner (e.g., as a specific-purpose computer) in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

Ultrasound imaging is an important tool for diagnosing a wide range of medical conditions. Examples of medical conditions that are diagnosable using ultrasound imaging include, by way of non-limiting examples, retinal detachments, pericardial effusions, acute appendicitis, nephrolithiasis, urinary retention, testicular torsion, deep vein thrombosis and muscle tear and rupture. Ultrasound imaging is also used, more broadly, in the fields of obstetrics (e.g., for fetal anatomy scans and automatic placenta localization), oncology (e.g., for assessing endometrial thickness and determining the need for endometrial biopsy), emergency medicine (e.g., for accurate and early diagnosis of acute cholecystitis), as well as for monitoring various chronic conditions (e.g., heart failure and diabetic complications such as peripheral arterial insufficiency).

Ultrasound scans, however, often require the presence of highly skilled technicians, known as sonographers, to operate complex ultrasound equipment. Many rural and remote communities often suffer from a lack of available sonographers. For patients in these communities, accessing sonographic services necessitates difficult, and often inconvenient trips to larger urban centers where these services are readily available. To this end, the lack of immediate access to ultrasound services often results in delays in medical diagnosis, as well as consequent delays in follow-up medical treatment, for patients living in these communities.

The requirement for an on-site ultrasound technician also limits accessibility to sonographic services in other contexts. For example, accessing ultrasound services is limited, if not entirely unavailable, in many long-haul trips (i.e., trips undertaken via ships, aircrafts, etc.). As well, as deep space exploration becomes a reality—the requirement for on-site sonographers presents a barrier to accessing important diagnostic ultrasound services for humans situated in space.

While mobile diagnostic imaging may increase the accessibility of ultrasound services, this approach fails to fully address the issue of accessibility of diagnostic quality ultrasound. More particularly, mobile diagnostic imaging services involve transporting mobile ultrasound imaging equipment, and skilled sonographers, to remote communities. These services, however, are hampered by the difficulty in recruiting expert staff that are able and willing to work in these remote communities. Additionally, these services suffer from high operating costs, as well as complex logistics in coordinating staff to work in remote communities.

The present disclosure provides systems, methods and devices for remote ultrasonography. The disclosed systems and methods can mitigate at least some of the drawbacks inherent in existing systems, and thereby provide improved access to diagnostic quality ultrasounds.

As described in further detail herein below, a remote ultrasonography system can include a local control system and a remote ultrasound system. The local control system can be located, for example, in an area accessible by skilled sonographers (e.g., an urban center). In contrast, the remote system can be located in a peripheral site, which is geographically remote to the local system. This may include, for example, a remote community, or otherwise a transportation ship, an aircraft, a space station, or any other desired environment.

The remote system can include one or more robotic devices operable to perform ultrasound scanning. The robotic devices can be installed, for example, in a dedicated scanning room in the remote site. The robotic devices can include one or more robotic arms. The robotic arms can retain tools required for ultrasound scans (e.g., gel dispensers, ultrasound transducers, etc.), and translate these tools over the patient's body as required. The remote ultrasound system can be used to capture sonograms of a patient, and may transmit these sonograms back to the local control system. The captured sonograms may then be reviewed locally by a sonographer, and further analyzed by a medical expert (e.g., a radiologist).

The remote robotic system can be configurable in one of three modes of operation: (i) a non-autonomous or guided mode; (ii) a fully autonomous or non-guided mode; and/or (iii) a semi-autonomous or semi-guided mode.

In a “non-autonomous” mode, the robotic system can be entirely guided by the local control system. For example, an input device, of the local control system, may guide motion of the remote robotic arm. In this manner, a local system operator (e.g., a skilled sonographer) can control the remote robot arm to pick-up and exchange various tools, apply gel to a desired location on the patient, move and re-orient ultrasound transducers to capture sonograms, etc. The local operator may also control image capture using the ultrasound transducer, and configure ultrasound parameters to obtain enhanced sonographic images.

The remote system can be enabled with haptic feedback. Through haptic feedback, the local user operator may receive feedback in respect of force and pressure experienced by the remote robotic arm. In turn, this may assist the local user operator in avoiding application of undue or excessive pressure to the patient. Haptic feedback can also assist in some types of medical diagnostics.

In contrast, in the “fully autonomous” mode, all functions of the remote system are fully automated, including gel application, ultrasound scanning, image capture, and preliminary medical prediction of the diagnosis.

To this end, to enable fully automated scanning, the remote system may host one or more trained machine learning models. The machine learning model can be trained to identify irregularities in an organ of interest based on sonographic data generated by an ultrasound transducer. The remote system may then control and adapt itself to capture more optimized images of the irregularity, and otherwise modify the ultrasound transducer position and configuration parameters to obtain the enhanced images. The captured images may then be transmitted back to the local system for preliminary review by an ultrasound technician, and further analyzed by a radiologist.

In a fully automated mode, the remote system may also perform automatic preliminary diagnosis of a patient. For example, the remote system may host a machine learning model trained to analyze irregularities in sonograms and provide preliminary diagnoses of associated medical conditions (e.g., in real-time or near real-time). The preliminary diagnoses may then be subsequently validated and/or confirmed by a medical expert (e.g., a radiologist).

In a “semi-autonomous guided” mode, the remote system may perform at least some functions under the control (or guidance) of the local control system, and may perform other functions autonomously and independently. For example, motion of the robotic arm may still be controlled by the local control system, however, the remote system may automatically control the robotic arm to avoid obstacles and prevent collisions. In other cases, the local operator may control the robot arm to capture sonograms, but the robot may otherwise automatically analyze the captured sonograms to provide preliminary diagnosis.

The remote system can be configured to dynamically vary between different modes of operation. For example, this may occur in response to receiving a user input to change the operation mode. The user input may be received at the local control system, or directly at the remote system.

In other cases, the remote system may change its mode of operation based on a trigger event. For example, the system may initially operate in a guided, non-autonomous mode. However, in response to detecting connection latency, the remote system may automatically revert to a semi-guided mode, or a fully autonomous mode.

In the guided and semi-guided modes, the remote system can be deployed at far distances from a skilled technician. This can extend sonographic services to areas located remotely from skilled sonographers, i.e., remote communities. In turn, this minimizes the “hidden tax” on patients residing in rural and/or under-serviced localities and improves access to diagnostic quality ultrasounds for these communities.

The non-autonomous or guided mode requires only a single sonographer to perform an ultrasound. This avoids the need for a healthcare professional to be attendant on-site at the remote system. This simplifies deployment logistics and reduces costs associated with hiring additional medical professionals.

In the autonomous mode, the need for a skilled practitioner may be entirely removed at both the control system end, and at the remote system end. Use of the autonomous mode can address important issues associated with communication latency. In particular, operation in the autonomous mode is not contingent on a stable network connection between the remote system and a sonographer at the local system. Accordingly, in areas having poor connectivity (e.g., remote communities, deep space exploration, etc.), the autonomous mode can enable for full operation of the ultrasound machine, including automated capture of sonograms and/or analysis of sonograms to provide preliminary diagnosis.

### Remote Ultrasound System

The following is a discussion of an example remote ultrasound system, which may be used alone or in any combination or sub-combination with any other feature or features described herein (e.g., an input device, a local user control system, a remote robotic control system, the methods for operating a remote ultrasound system etc.).

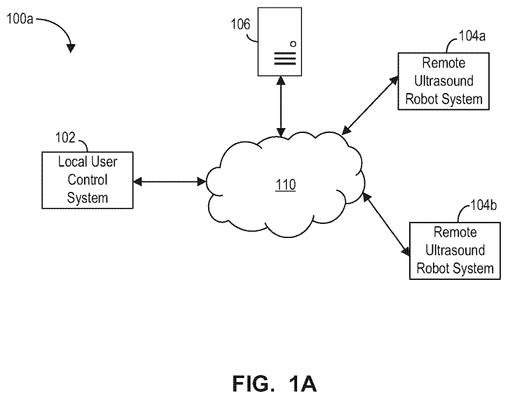

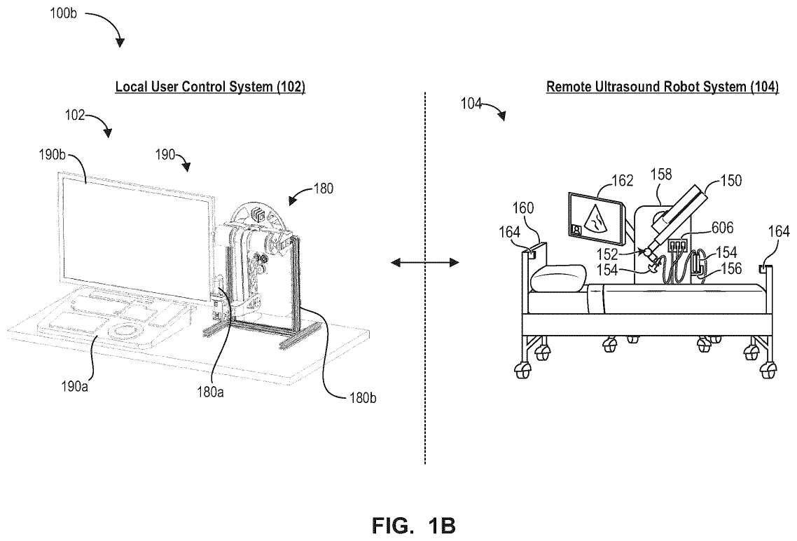

Reference is now made to FIG. 1A, which shows a simplified block diagram of an example system 100a for remote ultrasound scanning.

As shown, the system 100a can include a local control system 102 (also referred to as a local user control system) connected, via a network 110, to one or more remote robotic ultrasound systems 104a, 104b.

Local system 102 may control, or otherwise interact, with the remote systems 104. The local system 102 may be operated by a skilled technician or sonographer. The local system 102 may be located, for instance, in an urban center having a high availability of skilled sonographers.

Remote ultrasound systems 104 can be located remotely from the local system 102. The remote systems 104 can be installed in various remote, rural communities. In other example applications, the remote system 102 are installed in any other remote, mobile or non-mobile environment (e.g., an aircraft, a ship, a space station, etc.). To this end, the remote systems may be installable in a scanning room, or a specialized room, located in the remote areas. While only two remote systems 104 are exemplified, it will be understood that the system 100a can include any number of remote systems 104.

It should also be appreciated that remote ultrasound systems 104 need not be located in remote or rural locations, but rather that they are not at the same location as the local control system 102. For example, a remote ultrasound system 104 may be located within the same venue (e.g. a hospital) as the local control system 102 but in a different room or wing from the local control system 102.

As explained herein, each remote system 104 may be operated to capture sonograms of patients situated in the respective remote areas. The sonograms are then transmitted back to the local system 102 and/or server 106, and may undergo further review by local experts (e.g., sonographers, radiologists, etc.). Accordingly, ultrasound services may be provided on-demand to remotely situated patients, thereby expediting medical diagnostic assessments for these patients.

Optionally, the remote system 104 may be operable in one of three modes of operation, which include: (i) a non-autonomous mode, (ii) a semi-autonomous mode, and/or (iii) a fully autonomous mode.

In particular, as noted above, in the non-autonomous mode, operation of the remote system 104 is entirely guided by the local system 102. In the semi-autonomous mode, the remote system 104 may be partially controlled by the local system 102, and may be partially automatedly controlled. In the fully autonomous mode, the remote system 104 may operate entirely independently of the local system 102. In particular, in the fully autonomous mode, it is not necessary for the remote system 104 to be connected to the network 110.

As provided herein, system 100a enables a single sonographer-operating the local system 102—to remotely control multiple remote systems 104. Accordingly, a single sonographer may provide sonographic services to a plurality of remote systems located in the same or different geographic areas. In turn, this avoids the need to provide separate sonographers in each remote area. Accordingly, diagnostic quality ultrasounds may be provided to many remote locations in a scalable and economically sustainable manner.

In other example cases, the system 100a may also include more than one local system 102. For example, a separate local system 102 may be provided for each remote system 104.

Network 110 may be connected to the internet. Typically, the connection between network 110 and the Internet may be made via a firewall server (not shown). In some cases, there may be multiple links or firewalls, or both, between network 110 and the Internet. Some organizations may operate multiple networks 110 or virtual networks 110, which can be internetworked or isolated. These have been omitted for ease of illustration; however, it will be understood that the teachings herein can be applied to such systems. Network 110 may be constructed from one or more computer network technologies, such as IEEE 802.3 (Ethernet), IEEE 802.11 and similar technologies.

System 100a may also include one or more servers 106 connected to network 110 (although only one is shown for simplicity). Servers 106 may include, for example, virtual or “cloud” servers.

Servers 106 can perform various functions in the system 100a, such as to provide a storage database for images (i.e., sonograms) captured by remote system 104. The database can be accessible by a computer of the local system 102 (or any other computer). Sonograms captured by the remote system 104 in a semi-autonomous or fully autonomous mode may be uploaded to the servers 106. At a subsequent point in time, a sonographer can review the captured images to provide image quality control. The images may also be accessed and reviewed by a medical expert (e.g., a radiologist) to provide diagnostic assessments of imaged irregularities. Servers 106 may also store, in associations with sonographic images, various preliminary diagnostic assessments generated automatically by the remote system 104.

Servers 106 can also store an action log of various commands and data transmitted and received between the local and remote systems 102, 104. The action log may be used to monitor and/or audit system performance.

Reference is now made to FIG. 1B, which shows a schematic illustration 100b of an example portion of the system 100a. More particularly, FIG. 1B illustrates an example of a local system 102 and a remote system 104.

As shown, local control system 102 includes various features which allow a local operator (e.g., a skilled sonographer) to interact with the remote ultrasound system 104. For example, the local control system 102 may include an input device 180 and a computer workstation 190.

Input device 180 enables user control of a motion subsystem in the remote environment. To this end, the remote system 104 may include a motion subsystem which controls the position of tools (e.g., gel dispensers, ultrasound transducers, etc.), relative to a patient. The remote motion subsystem may include, for example, one or more robotic arms 150 with an integrated end effector 152 usable to retain (and manipulate) tools. Alternatively, the motion subsystem can include a gantry type system for example.

In operation, the input device 180 may allow a local operator (e.g., a sonographer) to control the remote motion subsystem, and in turn, vary a position of the tool-carrying end effector 152. For example, the local operator 180 can use the input device 180 to translate an ultrasound transducer over a patient in the remote environment.

As provided herein, positional control by the input device 180 can include spatial positional control (e.g., X, Y, Z position control), as well as orientational positional control (e.g., roll, yaw and pitch control) of the remote end effector 152. In general, the input device 180 can be used to control the pose of the end effector 152 and a tool retained by the end effector 152. The input device 180 may be used, for example, when the remote system 104 is operated in either the guided mode or semi-guided mode.

Any suitable input interface 180 may be provided in the local system 180. As shown in the example illustrated, the input device 180 may include a movable joystick arm 180a, which is supported by a frame 180b. Accordingly, a local sonographer may manipulate the spatial and orientational position of the joystick 180a. In turn, the local system may track the changing position of the joystick 180a and may generate control instructions (also referred to herein as “motion demands”) to effect reciprocal control of the remote motion subsystem.

Optionally, input device 180 may also receive data back from the remote system 104. For example, input device 180 can be configured to mirror forces and pressures experienced by the remote system 104 such as to provide a haptic feedback effect. For example, these can include forces experienced by the remote motion subsystem when colliding with obstacles in the remote environment (e.g., the patient's body or an external object). Haptic feedback may assist the local operator in ensuring that that they are not applying undue, or excessive force to the patient during ultrasound scanning, i.e., when operating the remote motion subsystem.

Alternatively or in addition, the input device 180 may include any other suitable interface for receiving inputs. For example, the input device 180 can be any other type of Cartesian style input device and can include any suitable parallel manipulator configuration design. In still some other example cases, input device 180 may include a virtualized computer interface (e.g., a touchscreen display) which allows the operator to manipulate a remote motion subsystem via input computer commands.

Local system 102 can also include the computer workstation 190. Computer workstation 190 may enable a user operator to input additional control data for controlling the remote system 104. For example, the workstation 190 can include an input interface 190a, such as a keyboard and/or mouse. The keyboard may be a general keyboard and/or a specialized ultrasound keyboard. The input interface (e.g. keyboard 190a) can be used to adjust various ultrasound parameters of an ultrasound transducer in the remote environment. The input interface 190a can also be used, for example, to instruct the remote system to capture images, exchange or swap an actively used tool (e.g., swap between different ultrasound transducers), adjust camera toggle for various cameras located in the remote system 104, pan, tilt or zoom the cameras located in the remote system 104 etc.

In some example cases, the input interface 190a may be at least a partially virtual computerized interface. For example, the input interface 190a may comprise a touchscreen display or virtual keyboard, which can receive various inputs.

Workstation 190 can also include a display interface 190b. Display interface 190a allows the local operator to view ultrasound images received from the remote system 104. For example, this can include viewing a real-time or near real-time feed from an actively deployed ultrasound transducer. In other cases, the display 190 can be used to view previously acquired sonograms (e.g., stored on server 106).

Display interface 190a can also display a video or image feed from one or more cameras 164 positioned in the remote environment. This, in turn, can enable the local operator to better monitor the remote environment, as well as to observe the remote patient.

Optionally, workstation 190 can, itself, include a camera and/or a microphone. The camera and/or microphone can allow the local operator to interact with the remotely-situated patient. For example, the local operator can use the camera and microphone to instruct the patient in respect of changing their pose, so as to facilitate ultrasound scanning.

The computer workstation 190 may be coupled to the input device 180, and may serve as the communication intermediary between the input device 190 and the robot system 104. For example, the computer 190 may receive control data, generated by the input device 180, and may transmit this data to the robot system 104. In the reverse case, the computer 190 may receive current position data from the robot system 104, and may transmit this data to the input device 180 (e.g., to implement haptic feedback). Alternatively, the input device 180 may be a stand alone device, and may include its own communication interface for independent communication with the remote system 104.

Turning now to the remote system 104—as stated previously, the remote system 104 may include a motion subsystem for retaining various ultrasound tools, and to manipulate the position of these tools relative to the patient. The motion subsystem may be entirely, or at least partially guided, by control instructions received from input device 180. Alternatively, the motion subsystem may operate autonomously, e.g., to independently capture sonographic images of a patient.

The motion subsystem can include a controllable robotic arm 150. The robotic arm 150 may include a distal end effector 152, which retains and exchanges various tools 154 (e.g., a gel dispenser, an ultrasound transducer probe, etc.). The robotic arm 150 may be positioned over a patient who is lying, for example, in the supine position on bed frame 160.

While only a single robotic arm 150 is exemplified, it should be understood that the system may include any other number of robotic arms 150. Alternatively or in addition, the motion system can include any other suitable motion mechanism, such as an XY gantry system.

As explained herein, the remote system can also include one or more object tracking sensors (e.g., LiDAR or depth-sensing cameras), as well as cameras 164 and a display interface 162. Cameras 164 can capture a live video and/or audio feed of the patient area. This feed can be transmitted back to the local user system 102. In this manner, a local system operator (e.g., a sonographer) can monitor the patient, as well as to observe and track movement of the motion subsystem (e.g., robotic arm 150).

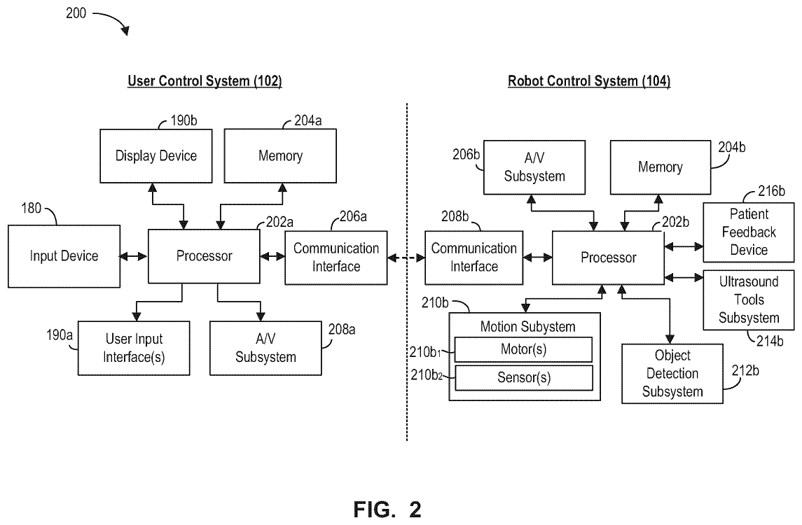

Reference is now made to FIG. 2, which shows a simplified electrical hardware block diagram 200 of an example of each of the local system 102 and the remote system 104.

As shown, the local user system 102 may include a processor 202a which is coupled, via a data bus, to one or more of the user input interface 190a, the display device 190b, the input device 180, as well as an audio/video (A/V) subsystem 208a and a communication interface 212a.

Processors 202a, as well as components 204a-210a may be housed within the computer workstation 190.

Processor 202a can be a computer processor, such as a general purpose microprocessor. Alternatively, processor 202a may be a field programmable gate array, application specific integrated circuit, microcontroller, or other suitable computer processor.

Processor 202a can be coupled, via a computer data bus, to memory 204a. Memory 204a may include both volatile and non-volatile memory. Non-volatile memory stores computer programs consisting of computer-executable instructions, which may be loaded into volatile memory for execution by processor 202a as needed. It will be understood by those of skill in the art that references herein to the local system 102 as carrying out a function or acting in a particular way imply that processor 202a is executing instructions (e.g., a software program) stored in memory 204a and possibly transmitting or receiving inputs and outputs via one or more interface. Memory 204a may also store data input to, or output from, processor 202a in the course of executing the computer-executable instructions.

Memory 204a may store various programs and algorithms which enable conversion of input data, generated by the input device 180, into control instructions to be transmitted to the remote system 104. In other cases, memory 204a may store programs and algorithms which convert position data, received from remote system 104, to control data for the input device 180.

Communication interface 206a is one or more data network interface, such as an IEEE 802.3 or IEEE 802.11 interface, for communication over a network (e.g., network 110).

Audio/visual (A/V) subsystem 208a can include one or more cameras, microphones and speakers. The A/V system 208a may allow the local user operator to interact and communicate with the remote patient, e.g., to provide instructions during scanning operations.

Input device 180 may include any interface for generating motion commands for a remote motion subsystem. While not shown in FIG. 2, the input device 180 may include its own processor, memory and communication interface, independent of the processor 202a, memory 204a and communication interface 206a.

User input interface 190a can include any interface for receiving user inputs, including keyboards and mouses. As noted above, the input interface 190a can include, for example, an ultrasound keyboard.

Display device 190b can be any suitable display for outputting information and data as needed by various computer programs (e.g., a desktop monitor). In particular, display 190b may display a graphical user interface (GUI). In some cases, the display interface 190b may include a touchable screen, and may therefore integrate the user input interface 190a.

Continuing with reference to FIG. 2, the remote control system 104 may also include a processor 202b coupled, via a data bus, to one or more of a memory 204b, an audio/visual (A/V) subsystem 206b, a communication interface 208b, a motion subsystem 210b, an object detection subsystem 212b, ultrasound tools subsystem 214b and/or a patient feedback device 216b.

Processor 202b, memory 204b and communication interface 208b may be analogous to processor 202a, memory 204a and communication interface 206a, respectively.

To this end, to will be understood by those of skill in the art that references herein to the remote system 104 as carrying out a function or acting in a particular way imply that processor 202b is executing instructions (e.g., a software program) stored in memory 204b and possibly transmitting or receiving inputs and outputs via one or more interface.

Memory 204b can store various programs used for controlling the motion system 210b. Memory 204b can also store one or more trained machine learning models. The trained machine learning models can be operable to control the motion subsystem 210b to automatically move various ultrasound tools to automatically capture sonographic images of irregularities detected in the patient's body. The trained machine learning models may also be used to automatically analyze irregularities in captured sonograms to provide a preliminary diagnosis of a patient's medical condition. The trained machine learning models may therefore enable operation of the remote system 104 in a fully autonomous, or semi-autonomous mode.

Audio/Visual (A/V) subsystem 206b may include, for example, one or more monitors (e.g., monitor 162 in FIG. 1B), speakers and microphones. The A/V subsystem 206b allows a patient to receive a video feed from the remotely situated sonographer, such as to allow the sonographer to interact with the patient.

Motion subsystem 210b can include any suitable system for retaining and translating ultrasound tools. For example, the motion subsystem 210 can include one or more movable robotic arms 150 (FIG. 1B). To this end, the motion subsystem 210b can include various controllable motors 210b₁ for motion control. Motion subsystem 210b can also include sensors 210b2. Sensor 210b₂ can include various position tracking sensors (e.g., encoders), as well as force sensors. Force sensors can be used to monitor pressure and force experienced by the motion subsystem, such as to enable haptic feedback at the input device 180.

Object detection subsystem 212b can include various depth-perception cameras and sensors (e.g., time of flight sensors, such as LiDAR). As explained herein, the object detection subsystem 212b can be used to monitor for obstacles obstructing the path of the motion subsystem 210b.

Ultrasound tools subsystem 214b can include various electronic hardware required to operate tools required for ultrasound scanning. For example, this can include drivers for ultrasound transducers, pumps for operating gel dispensers, etc.

Optionally, the remote system 104 may also include a patient feedback device 216b. The patient feedback device 216b can include a pain threshold monitor, which can be used by a patient to indicate if the remote system 104 is applying undue or excessive pressure. An input from the input device 216b may cause the system to disengage, or otherwise, decrease the applied pressure.

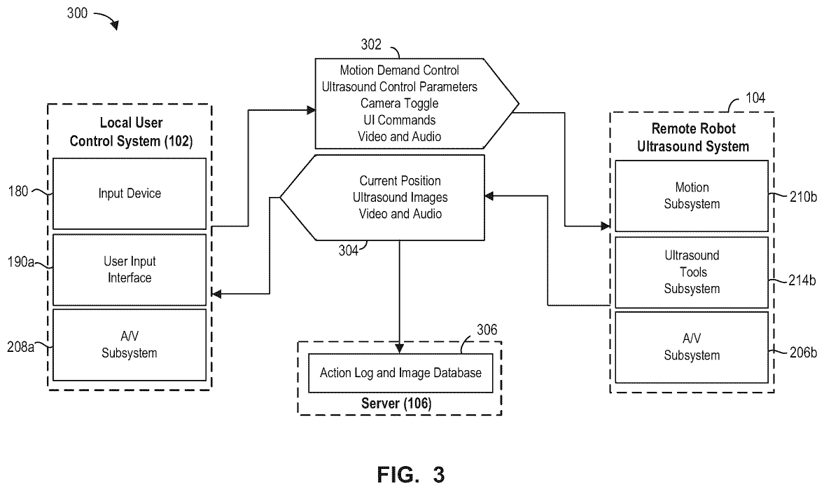

Reference is now made to FIG. 3, which is a simplified block diagram 300 illustrating data flow between a local user control system 102 and a remote robot ultrasound system 104.

As shown, the local control system 102 can generate and transmit, to the remote system 104, various user-generated data 302. The user-generated data 302 can include motion demand control, generated by the input device 180. Motion demand data can be used for controlling and manipulating the remote motion subsystem 210b (e.g., the robot arm 150).

The user-generated data 302 can also include various data generated by the user input interface 190a, including, (i) ultrasound control parameters for controlling ultrasound transducers in the remote environment (e.g., generated using an ultrasound keyboard); (ii) camera toggle for cameras in the remote system 104; (iii) camera adjustments (e.g. pan, tilt zoom) for cameras in the remote system 104; as well as (iv) other user input commands (e.g., controlling tool exchange switching by the motion subsystem 210b, operating tools, varying the operational mode of the remote system 104, etc.). User-generated data 302 may also include various audio and visual data generated by the A/V system 208a. For example, this can include images and speech generated as the local operator interacts with the remote patient.

Remote system 104 may receive the user-generated data 302, and in response, may control the motion subsystem 210b as well as the various ultrasound tools 214b. The remote system 104, in turn, may generate and transmit its own remote system data 304. For example, this can include current position data in respect of the current position of the motion subsystem 210b. This may be determined, for example, based on senor data generated by the sensors 210b₂ of the motion system 210b (FIG. 2). Remote system data 304 can also include ultrasound images generated by the ultrasound tools 214b (e.g., ultrasound transducers). In various cases, video and audio data 304 may also be transmitted, which includes video and audio of the patient interacting with the remote sonographer.

In at least some cases, the remote system data 304 may also be transmitted to the server 106. For example, server 106 may store an action log, as well as a database of various captured images.

### General Description of an Input Device

The following is a discussion of an example input device which may be used alone or in any combination or sub-combination with any other feature or features described herein (e.g., a remote ultrasound system, a local user control system, a remote robotic control system, the methods for operating a remote ultrasound system etc.).

An input device is described herein, which may be used by itself, as part of the local control system 102, or otherwise in conjunction with one or more components of system 100a in FIG. 1A.

As provided above, the input device 180 enables control of the motion subsystem 210b in the remote environment. Motion subsystem 210b can include, for example, a remote robotic arm 150, or otherwise any other suitable system for retaining and moving tools in the remote environment for the purposes of ultrasounds scanning (e.g., a gantry type system).

As provided herein, the disclosed input device 180 provides one or more of spatial and orientational control of an end effector of the motion subsystem 210b. The end effector is the portion of the motion subsystem which retains tools used for ultrasound scanning.

More particularly, spatial control enables the input device 180 to control translation of the end effector to any spatial direction (e.g., along an X, Y, Z axis defined in the remote reference frame). In turn, spatial control allows a local operator, for example, to move a gel dispenser or an ultrasound transducer to different positions over a patient's body. Orientational control enables the input device 180 to control the pitch, yaw and roll of the remote tool-retaining end effector. This may, for example, allow the local operator to roll, rock or rotate an ultrasound transducer attached to the end effector (e.g., to obtain enhanced sonographic images).

In the example illustrated, the input device 180 includes a user-movable arm system. The arm system is movable to different spatial and orientational positions by the local operator (e.g., a sonographer). In turn, movement of the arm system enables corresponding control of the motion subsystem 210b, in the remote environment. The arm system can include a joystick (also referred to herein as a “dummy probe”), which provides a simplified engagement interface for the user to engage the arm system.

To this end, the arm system includes one or more arm segments in rotatable connection. The arm segments may provide up to six degrees of freedom of motion, in both the spatial and orientational dimensions. In the example illustrated, the rotational connection between arm segments is provided by various shafts, as well as belt and pulley mechanisms.

As exemplified herein, input device 180 may also include a sensor subsystem. The sensor subsystem may be used to sense and track positional movement of the arm system. Data generated by the sensor subsystem can be used to generate motion demand controls for controlling the remote motion subsystem 210b. The sensor subsystem 212a can include, for example, one or more encoders (e.g., rotary and/or linear encoders). The encoders may be coupled to the rotatable shafts, or belt and pulley mechanisms, to track rotation and motion of these mechanisms.

Input device 180 may additionally provide for haptic feedback functionality. Haptic feedback enables the input device 180 to mirror forces and pressures experienced by the motion subsystem 210b in the remote environment. As provided herein, the haptic feedback may be effected by one or more high-torque DC motors of the input device 180. The DC motors may couple to rotatable shafts, and may apply counter rotational force to simulate the effect of resistance. The DC motors can additionally stabilize the arm system in static position against the force of gravity.

To this end, the haptic feedback functionality has a number of appreciated advantages: (i) first, haptic feedback ensures that the user is prevented from moving the input device 180 to a positional state that is otherwise inaccessible in the remote environment to the motion subsystem 210b (e.g., as a result of an obstacle in the path of the motion subsystem 210b). In turn, the input device 180 may more closely track motion in the remote system 104; (ii) second, certain diagnostics may be facilitated by monitoring different levels of pressure applied to the patient's body, which is facilitated by the haptic feedback; and (iii) third, the local operator is prevented from applying excessive pressure to the patient, which may otherwise injure the patient.

Optionally, input device 180 can also include an activation toggle. The activation toggle can operate as an input device clutch to connect or disconnect the motion of the input device 180 from the motion subsystem 210b. The activation toggle can be mounted directly to the input device 180 (e.g. joystick 450) to allow an operator to easily adjust the activation toggle while manipulating the input device 180. Alternatively or in addition, an activation toggle may be provided by the user input interface 190a.

The activation toggle can be adjusted between an active position and an inactive position. The activation toggle may be a contact sensor, a soft toggle (e.g., a touch sensitive area of a touchscreen on the input device 180) or a physically moveable toggle such as a slider, a pivoting switch, a depressible button such as a trigger etc.

When the toggle is in the active position, motion of the input device 180 can be connected to the motion subsystem 210b such that positional movement of the arm system is used to generate motion demand controls for controlling the remote motion subsystem 210b. When the toggle is in the inactive position, motion of the input device 180 is disconnected from the motion subsystem 210b such that positional movement of the arm system does generate motion demand controls for controlling the remote motion subsystem 210b. This may allow an operator to re-position or reset the input device 180 in order to rest or re-adjust to a more comfortable control position.

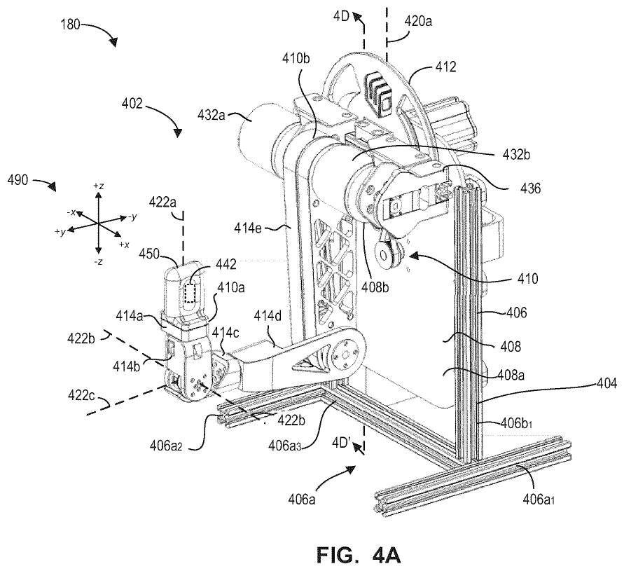

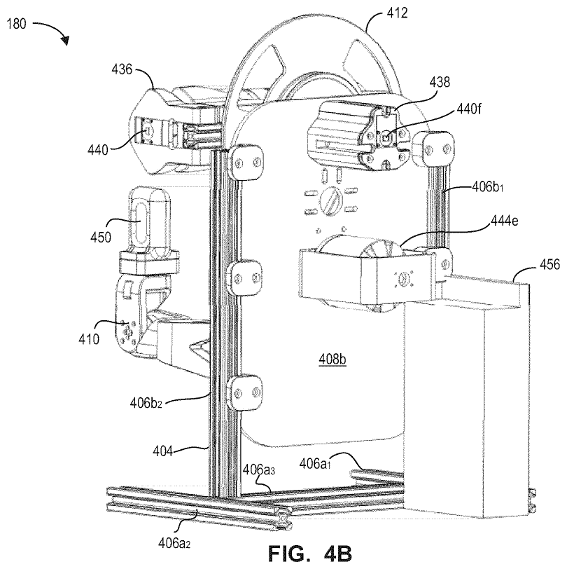

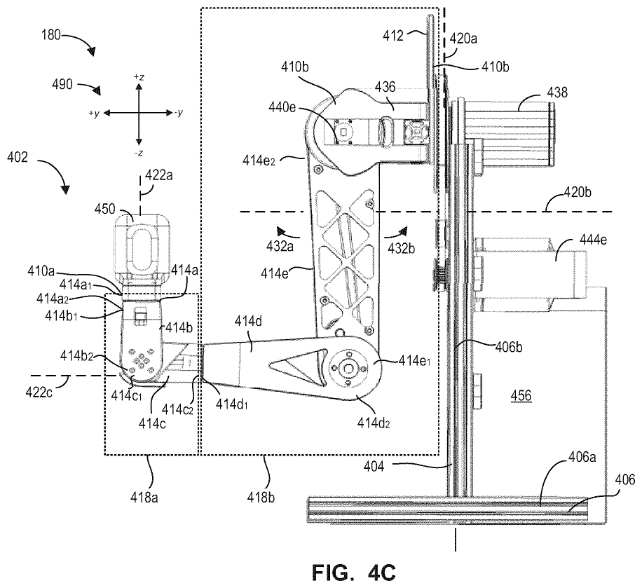

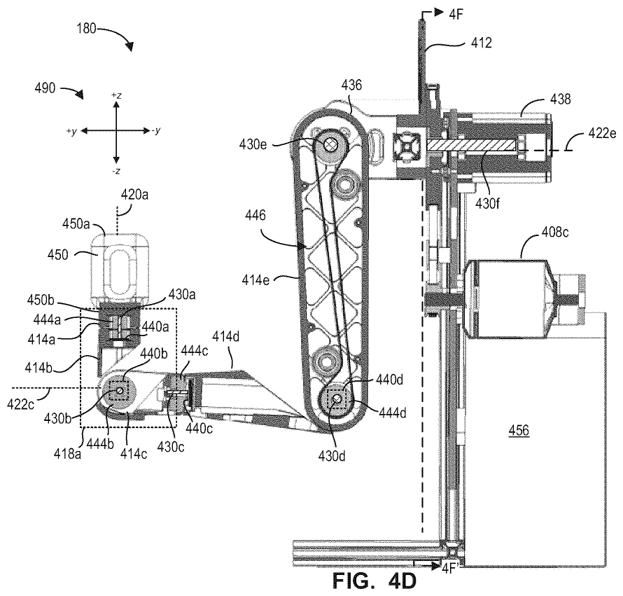

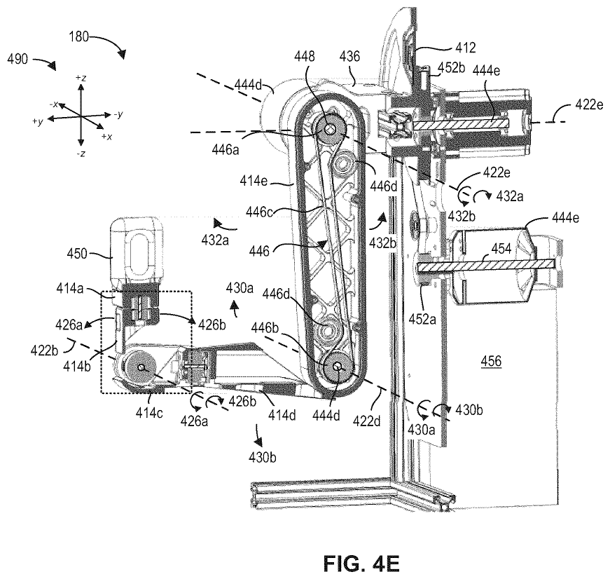

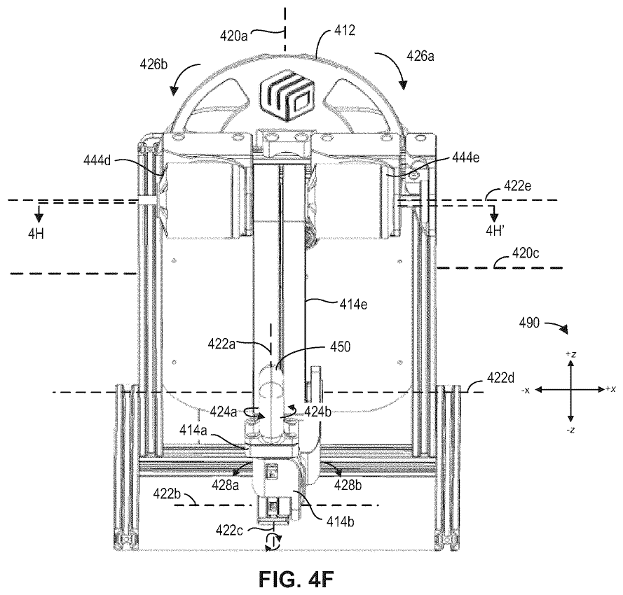

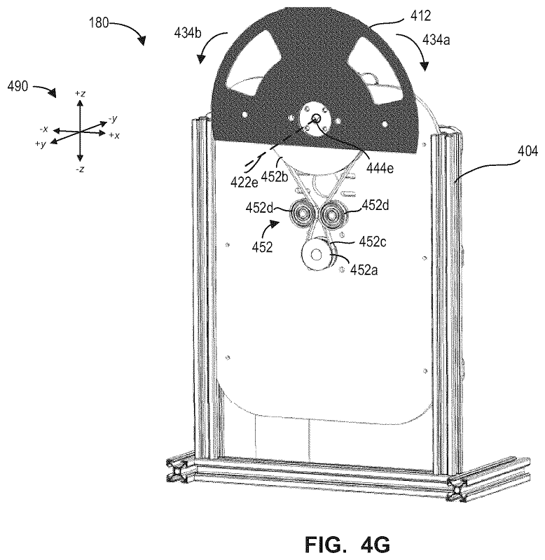

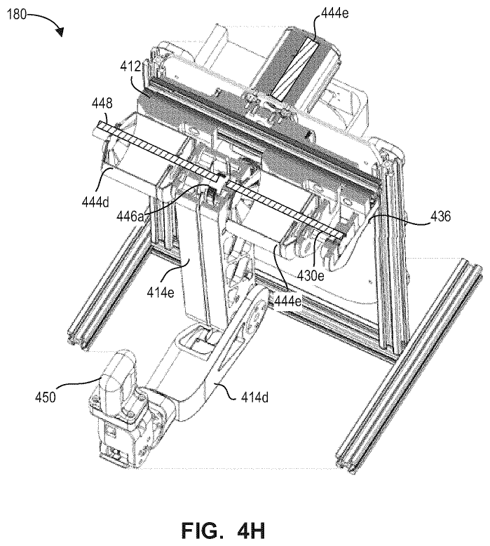

Reference is now made to FIGS. 4A-4F, which exemplify an example of an input device 180.

As shown, the input device 180 may include a user-controllable arm system 402 supported to a mounting structure 404.

As illustrated, arm system 402 includes a mechanical arm 410 extending between a first arm end 410a, and a distal second arm end 410b (FIG. 4A). As shown, the mechanical arm 410 may extend between the first and second ends 410a, 410b along a horizontal longitudinal axis 420b (FIG. 4C).

The mechanical arm 410, itself, can be mountable to a rotatable member 412. In the example illustrated, the rotatable member 412 includes a rotatable half-disk (FIG. 4G). Alternatively, the rotatable member 412 may have any other suitable shape or design. Rotatable member 412, in turn, can be secured to the mounting structure 404 (i.e., rotatably secured).

As provided herein, the combination of the mechanical arm 410 and rotatable member 412 enables a user to control both the spatial and orientational position of a tool-retaining end effector in the remote motion subsystem 210b. In the example illustrated, the mechanical arm 410—in conjunction with the rotatable element 412—provides up to six degrees of freedom of motion control (i.e., spatial and rotation control).

The first end 410a, of the mechanical arm 410, can be coupled to a joystick-type device 412 (also referred to herein as a “dummy probe”). Joystick 412 is engageable by a user to enable the user to move the arm system 402. Joystick 450 therefore acts as a “stand-in” for a tool located in the remote environment (e.g., a gel dispenser or an ultrasound transducer). Joystick 450 may include one or more contact sensors 442 (FIG. 4A). Contact sensors 442 can be used to monitor engagement of the joystick 412 by a user.

As further shown, second arm end 410b, of mechanical arm 410, is indirectly secured to the rotatable half-disk member 412. As explained, rotation of the half-disk member 412 allows corresponding rotation of the entire arm structure 410.

As further exemplified, mechanical arm 410 includes one or more arm segments 414 that are rotatably coupled. In the example illustrated, mechanical arm 410 includes five arm segments 414a-414e, in rotatable coupling (FIG. 4A). In combination, the arm segments provide up to five degrees of freedom of motion. Rotatable member 412 provides the sixth degree of motion freedom.

Alternatively, the mechanical arm 410 can be provided with a different number of arm segments, e.g. three arm segments. This may reduce the degrees of freedom of motion of the mechanical arm 410 while simplifying the control processing.

In more detail, with reference to FIG. 4C, the user-controllable arm system 402 may be segmented into two general portions: (i) a first portion 418a for orientational position control (i.e., an orientational-position control portion 418a), and (ii) a second portion 418b for spatial position control (i.e., a spatial-position control portion 418b).

Orientation control portion 418a enables the user to adjust the orientational position of the joystick 450. In turn, the user is able to adjust the orientation of a tool located in the remote environment. In the example illustrated, orientation control portion 418a provides up to three degrees of freedom of motion. These three degrees of motion correspond to the yaw, pitch and roll of joystick 450. Accordingly, a user may interact with the input device 180 to, for example, roll, rock or rotate an ultrasound transducer in the remote environment (e.g., to obtain enhanced sonographic images). In other cases, the portion 418a may provide three or less degrees of freedom of motion.

In contrast, spatial control portion 418b enables the user to adjust the spatial position of the joystick 450. The spatial control portion 418b may provide up to three degrees of motion freedom (i.e., X, Y and Z spatial movement). In turn, this may allow an operator to control the spatial position of a remote tool in the remote system 104. For example, the operator can move a gel dispenser or an ultrasound transducer to different positions over a patient's body.

Each of system portion 418a, 418b, is now discussed in greater detail herein, starting with the orientation control portion 418a.

Referring to FIGS. 4A and 4C, the orientation control portion 414a can include multiple arm segments of the mechanical arm 410. These segments include: (i) a forward “wrist” arm segment 414a, used for yaw or rotation control; (ii) a mid-arm rotation segment 414b, used for pitch or roll control; and (iii) a rear arm rotation segment 414c, used for roll control.

Forward wrist arm segment 414a enables controlling rotational orientation of the joystick 450. For example, as shown in FIG. 4F, this includes moving the joystick 450 in both the clockwise direction 424a, as well as counter clockwise direction 424b, about a first rotation axis 422a. First axis 422a may also be referred to herein as the “yaw” axis 422a, such that wrist arm segment 414a enables control of the joystick's “yaw” angle. In an initial position state, first axis 422b may be substantially aligned with a vertical axis 422a in the local environment (i.e., a z-axis defined in the local reference frame 490).

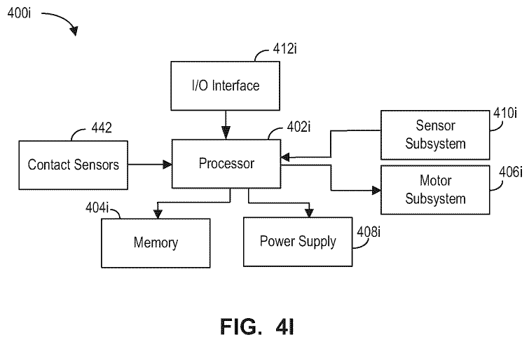

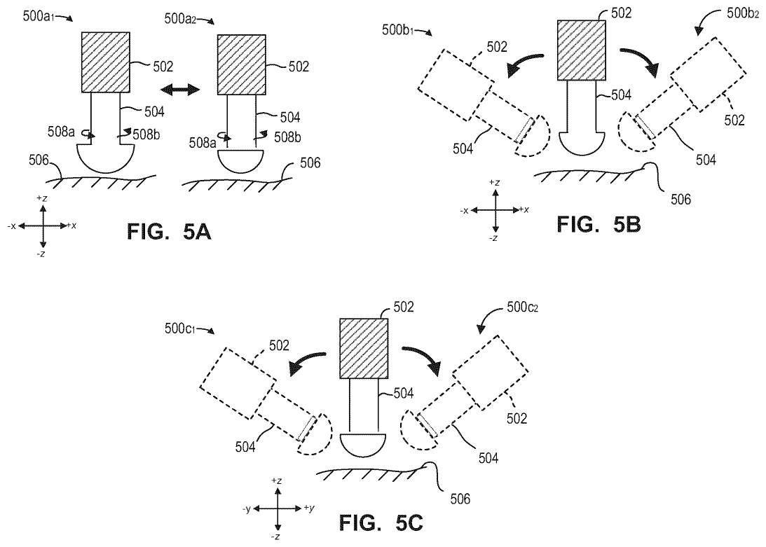

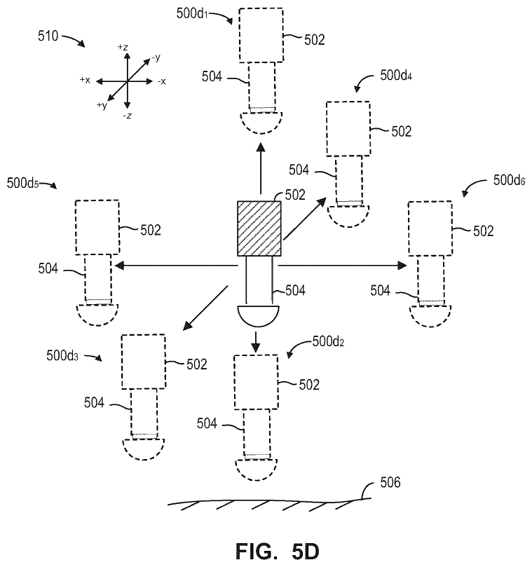

As shown in FIG. 5A, in the remote environment-rotation of the input device's wrist arm segment 414a may control corresponding rotation of an end effector 502 of the remote motion subsystem 210b. For example, the input device's arm segment 414a can control clockwise 508a (500a1) or counter clockwise 508b (500a2) rotation of the end effector 502. End effector 502 can be used to retain various tools, e.g., ultrasound transducers. In the case of an ultrasound transducer, rotation can enable the user to switch between viewing the long and short axis of a bodily structure or organ. The rotation can also be applied to any other tool coupled to the end effector 502.

As exemplified FIG. 4C, the input device's “wrist” arm segment 414a extends-along rotation axis 422a—between a first end 414a₁ and a second end 414a₂. First end 414a₁ can be rigidly connected to the joystick 450. First end 414a₁ also, more generally, defines the first end 410a of the mechanical arm 410. Second end 414a₂ can be rotatably connected to the mid-arm rotation segment 414b, and rotates about first axis 422a.

Turning now to the mid arm segment 414b—mid-arm segment 414b enables forward and backward rolling of joystick 450. For example, as shown in FIG. 4E, the mid-arm segment 414b may rotate about a second axis of rotation 422b, which is orthogonal to the first axis 422a. The mid-arm segment 414b can be rotatable in a counter clockwise direction 426a, to provide forward roll, and a clockwise direction 426b to provide backward roll. To this end, forward roll 426a may refer to rolling away from the mechanical arm 410, while backward roll 426b may refer to rolling towards the mechanical arm 410.

Second rotation axis 422b may also be referred to herein as the “pitch” axis 422b, such to enable controlling the “pitch” angle of the joystick 450. When the arm system 402 is in an initial position state, second axis 422b may be substantially aligned with a lateral horizontal axis 422a in the local environment (i.e., an x-axis defined in the local reference frame 490) (FIG. 4F)

Referring briefly to FIG. 5B, rotation of the mid-arm segment 414b enables corresponding rolling of end effector 502 in the remote environment. In this manner, an ultrasound transducer 504—coupled to end effector 502—can be rolled forward (500b1) and backward (500b2). In turn, this enables an operator to control “fanning” of the transducer over surface 506. As is known in the art, fanning of a transducer facilitates visualization of multiple cross-sectional images of an organ structure of interest.

As best shown in FIG. 4C, mid-arm segment 414b also extends between a respective first end 414b₁ and second end 414b2, and along an axis collinear with first axis 422a. First end 414b₁ can be rotatably connected to the forward wrist segment 414a to enable rotation about first axis 422a. Second end 414b₂ can be rotatably connected to the rear arm segment 414c, to enable rotation about second axis 422b.

Finally, rear arm segment 414c controls “rolling” of the joystick 450, or otherwise, left and right rolling of joystick 450. As exemplified in FIGS. 4A and 4F, rear arm segment 414b may rotate about a third axis of rotation 422c. The third axis 422c can be orthogonal to both the first and second axis 422a, 422b. Third rotation axis 422c may also be referred to herein as the “roll” axis 422b, such to enable controlling the “roll” angle of the joystick 450. When the arm system 402 is in an initial position state, third axis 422c may be substantially aligned, i.e., parallel, with the longitudinal horizontal axis 420c in the local reference frame (i.e., a y-axis defined in the local reference frame 490).

To this end, as shown in FIG. 4F, the rear arm segment 414c is rotatable about third axis 422c in a clockwise rotation 428a to facilitate leftward rolling, and in a counter clockwise rotation 428b to facilitate rightward titling 450, of the joystick 450.

As shown in FIG. 5C, rotation of the rear arm segment 414c provides for corresponding rolling of the end effector 502 in the remote environment. For example, the end effector 502 can be rolled, or rolled in the left (500c1) or right (500b2) direction. Where the end effector 502 retains an ultrasound transducer, this motion enables the operator to “rock” the ultrasound transducer. As is known in the art, rocking an ultrasound transducer assists in visually “centering” a desired area of interest. It will be noted that FIG. 5C is distinguished from FIG. 5B based on the axis in which the rolling occurs.

Referring back to FIG. 4C, rear arm segment 414c also extends between a respective first end 414c₁ and a second end 414c₂, along third axis 422c. First end 414b₁ can be rotatably connected to the mid-arm segment 414b, to enable rotation about second axis 422a. Second end 414b₂ can be rotatably connected to a translation arm segment 414d, to enable rotation about third axis 422c.

Accordingly, and in view of the foregoing, the three arm segments 414a-414c, forming the orientation control portion 414a, provide for full orientational control in any of the yaw, pitch and/or roll directions.

Reference is now made to the spatial-position control portion 418b of the arm system 402. Spatial control portion 418b includes, (i) a forward translational arm segment 414d; (ii) a rear translation arm segment 414e; and (iii) the rotating half-disk 412 (FIG. 4C).

The combination of these components enables a user to spatially move the joystick 450 in any X, Y, Z position within a local cartesian reference frame 490. In turn, the user is able to control the corresponding spatial position of a tool-retaining end effector 502 in a reference frame 510, defined with respect to the remote environment (FIG. 5D).

In more detail, forward translation arm segment 414d allows translating the joystick 450 upwardly and downwardly (FIG. 4C), i.e., along a vertical axis 420a (i.e., a z-axis defined in the local reference frame). To this end, arm segment 414d may rotate about a fourth rotation axis 422d (FIGS. 4F and 4E), which is parallel to the x-axis in the local reference frame. As shown in FIG. 4E, the arm segment 414d can rotate counter clockwise 430a about fourth axis 422d to move vertically upwardly, and clockwise 430b to move vertically downwardly.

As shown in FIG. 5D, an operator can use arm segment 414d to control the corresponding vertical position of the end effector 502 in the remote environment. That is, the operator is able to move the robot arm end effector 502 upwardly (500d1) and downwardly (500d2), along a z-axis defined in the remote reference frame 510.

In an example case where the end effector 502 retains an ultrasound transducer 504, the transducer 504 may be moved downwardly to bring the transducer closer to a contact surface 506 (i.e., a patient's body in the supine position) to generate sonographic images. In contrast, the tool 502 may be moved upwardly, to disengage from the contact surface 506.

Optionally, a transducer 504 can be moved downwardly to apply pressure to a contact surface 506. For example, this can increase the visualization area, and in turn, improve captured image quality. In other cases, downward pressure may be applied on one side of the transducer to angle the probe. In turn, this may allow the operator to direct or angle ultrasound beams in a desired direction. Still further, downward pressure can allow compressing a vein, or pushing an anatomical structure out of the way of an intended needle pass.

As shown in FIG. 4C, forward translation arm segment 414d extends between a first end 414d₁ and a second end 414d₂, along an axis collinear with the third rotation axis 422c. First end 414d₁ can be rotatably connected to the rear arm segment 414c and rotates about third rotation axis 422c. Second end 414d₂ can be rotatably connected to the rear translation arm segment 414e to allow rotation about fourth axis 422d.

Turning now to the rear translation arm segment 414e, this arm segment may allow translating the joystick 450 forwardly 432a and backwardly 432b (FIG. 4E). Forward and backward motion can be defined along a longitudinal horizontal axis 420c (i.e., defined along a y-axis in the local reference frame) (FIG. 4C). As used herein, the forward direction 432a may refer to a direction nearing the user operator, while the backward direction 432b may refer to a direction facing away from the user operator.

As shown in FIG. 5D, an operator can use rear arm segment 414e to control the corresponding lateral position of the end effector 502, in the remote environment. That is, the operator may translate the end effector 502 forwardly (500d3) or backwardly (500d4), along a y-axis defined in the remote reference frame 510. In turn, the tool 504 may be re-positioned at different positions along the contact surface 506. This enables spatially repositioning an ultrasound transducer or a gel dispenser.

Referring back to FIG. 4C, the rear translation arm segment 414e extends between a first end 414e₁ and a distal second end 414e₂. First end 414e₁ can be rotatably coupled to the forward translation arm segment 414d to enable rotation about the fourth rotation axis 422d (FIG. 4E). Second end 414e₂ can be rotatably mounted to enable rotation about fifth rotation axis 422e, such as to allow clockwise 432a and counter clockwise 432b rotation (FIG. 4E). In the example illustrated, the second end 414e₂ is rotatably mounted to a mounting bracket 436 (FIG. 4C), which is connected to the rotating half-disk 412.

Referring now to FIG. 4G, as illustrated the rotating half-disk 412 is rotatably mounted to the mounting structure 404, and rotates about a sixth axis of rotation 422e (FIGS. 4E and 4G). In the example illustrated, rotation axis 422e is colinear with the y-axis in the local reference frame 490 (FIG. 4E), as well as with the longitudinal horizontal axis 420c (FIG. 4C).

Rotation, of the rotatable half-disk 412 causes the entire mechanical arm 410—which is attached to the half-disk 412, via mounting bracket 436—to move in the right direction 434a and left direction 434b. In turn, this allows movement of the joystick 450 along the x-axis, defined in local reference frame 490.

As shown in FIG. 5D, rotation of half-disk 412 enables corresponding movement of the end effector 502 in the x-axis in the remote reference frame 510. In this manner, the operator can remotely translate a tool 504 in the right (500d5) and left (500d6) directions.

In view of the foregoing, the combination of the forward and rear translation arm segments 410d, 410e, along with the rotating half-disk 412, provides for full spatial control along any cartesian axis in the local and remote reference frames.

In the example illustrated, the rotatable connection, between the various mechanical arm segments 410—as well for the rotating half-disk 412—is implemented using rotating shaft members. The shaft members are disposed, for example, inside a hollow interior of each arm segment.

By way of example, as shown in FIG. 4D, a first shaft 430a rotatably connects the mid-arm segment 414b to the wrist arm segment 414a. First shaft 430a extends along first rotation axis 422a. Second shaft 430b rotatably connects the mid-arm segment 414b to the rear arm segment 414c, and extends along second rotation axis 422b. Third shaft 430c—extending along third axis 422c—rotatably connects rear arm segment 414c, to the forward translation arm segment 414d.

Similarly, a shaft 430d rotatably connects the forward and rear translation arm segments 414d, 414e together, and extends along fourth axis 422d. A further shaft 430e extends along the fifth rotation axis 422e (FIG. 4H), and rotatably connects the rear translation arm segment 414e to the mounting backet 436. Still further, a shaft 430f may extend along sixth rotation axis 422e, and may rotatably connect the rotating element 412 to the mounting structure 404. Shaft 430f may be larger in diameter, as it may sustain the weight of the entire movable arm 410. Optionally, shaft 430f is mounted inside of a shaft-receiving housing 438.

Alternatively, any other mechanism can be used for rotatably coupling various structural elements together.

As stated previously, a sensor subsystem of the input device 180 (FIG. 2) can monitor and track motion of the various arm segments 414, and rotating half-disk 412. In the illustrated example, the sensor subsystem includes a plurality of encoders 440 (e.g., rotary encoders). Each encoder may attach to an end of a rotating shaft 430, and may track rotation of that shaft in the clockwise and counter clockwise directions. For example, each shaft 430a-430f may have a corresponding rotary encoder 440a-440f coupled to one end of the shaft (see e.g., FIG. 4D, as well as FIGS. 4B and 4C).

Alternatively, the sensor subsystem can include an inertial measurement unit (IMU) coupled to joystick 450. The IMU can monitor the pose (position and orientation) of joystick 450.

Input device 180 may also include a plurality of motors 444 (e.g., direct current motors). Motors 444 may connect to one or more shafts 430a-430f, and may be used to drive the shafts within each rotatable connection.

In more detail, motors 444 may operate in one of two modes: (i) in a default mode, each motor 444 may run with low current, and apply only enough torque to hold each rotatable connection in static position. That is, each motor 444 may control a respective shaft 430 to maintain the arm segments 414, as well as the rotating member 412, in a static state. In turn, this prevents the movable arm 410 from collapsing under the force gravity when the user disengages the joystick 450; and (ii) in haptic feedback mode, a higher current may be applied to one or more of the motors 444. The higher current can be applied such that the motors 444 generate higher torque for a given shaft. In turn, the user is faced with increased resistance when attempting to move a rotatable connection in a given direction.

The default mode may be activated based on engagement or disengagement of contact sensors 442 in the joystick 450. For example, once the user disengages from the contact sensors 442, the motors 444 may be operated in the default mode to hold the arm system in position. In contrast, once the user engages the contact sensors 442, the motors 444 may be ready to operate in the haptic feedback mode. Alternatively or in addition, the default mode may be activated when the contact sensors 442 are engaged, such to assist the user in supporting the arm system against downward gravitational force.

As shown in the example illustrated, at least six motors 444a-444e can be provided. As shown in FIG. 4D, at least three motors 444a-444c are coupled to, and otherwise drive rotatable shafts 440a-440c, respectively. Motors 444a-444c may be disposed, for example, within a hollow interior of corresponding arm segments 414a-414d, respectively.

As best shown in FIG. 4F, an additional two motors 444d, 444e are also provided for controlling rotation of the front and rear translation arm segments 414d, 414e, respectively. To this end, as shown in FIG. 4H, motor 444e is connected to, and controls shaft 430e associated with the rear arm segment 414e.

Motor 444d may not directly connect to shaft 414d, which is associated with the front translation arm segment 414d. Rather, as best shown in FIG. 4E, motor 444d can be connected to shaft 444d via a belt and pulley mechanism 446. Optionally, the belt and pulley mechanism 446 extends through the hollow interior of the rear arm segment 414e. A larger motor may be required to control the weighty forward translation arm segment 414d. Owing to its size, this larger motor may not be receivable inside the arm segment (i.e., as shown with motors 444a-444c), and may therefore be positioned distally from arm segment 414d. The belt and pulley mechanism 446 therefore connects the distal motor 444d to drive front translation arm segment 414d.

In more detail, motor 444d can be connected to, and drive, a motor shaft 448. Motor shaft 448 is, itself, connected to a first pulley or gear 446a. Additionally, shaft 444d—which rotatable connects the front and rear arm segments 414d, 414e—can be connected to a second pulley (or gear) 446b. Belt 446c wraps around each of the gears 446a, 446b, and can be made sufficiently taught through one or more tension rollers 446d. In this manner, motor 444d may first rotate pulley 446a (via motor shaft 448), which in turn may cause rotation of second pulley 446b, and thereby rotation of the front translation arm segment 446b (via shaft 444d).

As best shown in FIGS. 4E and 4G, a similar belt and pulley mechanism 452 can be used to drive the rotatable half disk 412. For example, rotatable half disk 412 may be driven by motor 444e, which itself drives a corresponding motor shaft 454. Motor shaft 454 may rotatably engage, at one end, to pulley (or gear) 452a (FIG. 4E). A second pulley 452b may engage to the shaft 444e, which controls rotation of the rotatable half disk 412. A belt 452c may wrap around each of pulleys 452a, 452b, and can be made sufficiently taught through one or more tension rollers 452d (FIG. 4G). Accordingly, motor 444e may rotate pulley 452a (via motor shaft 454), which in turn may cause rotation of second pulley 452b, and thereby rotation of the half disk 412 (via shaft 444e).

Referring back to FIG. 4C, the input device 180 can also include a control box 456. Control box 456 may house, for example, a processor which is connected to each of the motors 444a-444e, various encoders 440, as well as joystick contact sensors 442. Control box 456 may also house a power supply for supplying power to motors 444.

As stated previously, to accommodate the weight of the movable arm system 402—the input device 180 may include the mounting structure 404. As best exemplified in FIG. 4C, mounting structure 404 may include a support frame 406. In the upright position, support frame 406 may include a base portion 406a and a vertical portion 406b coupled to the base. As shown in FIGS. 4A and 4B, each of the base and vertical portions 406a, 406b may include one or more respective support beams 406a₁-406a₃ (i.e., base portion), and 406b₁-406b₂ (i.e., vertical portion).

In the illustrated example, a mounting plate 408 is supported between vertical members 406a, 406b (FIG. 4B). Mounting plate 408 can be used for mounting the various components of the input device 180 (e.g., the movable arm 410, etc.). In other examples, mounting plate 408 may be integrally formed with the frame 406.

Reference is now made to FIG. 4I, which shows a simplified electrical hardware block diagram 400i for the input device 180.

As shown, the input device 180 may include an input device processor 402i coupled to one or more of a memory 404i, a motor subsystem 406i, a power supply 408i, a sensor subsystem 410i, an input/output (I/O) interface 412i and the one or more contact sensors 442 in the joystick 450.

Processor 402i and memory 404i may be analogous in architecture to processor 202a and memory 204a in FIG. 2.

Motor subsystem 406i may include the one or more motors 440a-440f, which are used to adjust the positional state of the arm system 402.

Power supply 408i may include, for example, one or more batteries which are used to power the motors 444. In other cases, power supply 406i may include an electrical power outlet. To this end, processor 402i may control the power supplied from the power supply 406i to the motors 444. For example, as noted previously, in a default mode of operation-processor 402i may supply a low current to each of motors 444. Otherwise, in a haptic feedback mode, processor 402i may supply a higher current to each of motors 444, as necessary (e.g., 0 to 60 Amps).

Sensor subsystem 410i may include one or more sensors for monitoring the positional state of the arm system 402. For example, these may include encoder 440a-440f.

I/O interface 412i may couple the input device 180 to workstation 190 (FIG. 2). The I/O interface 412i may enable the input device 180 to transmit encoder data, from encoders 440, to the workstation 190. In turn, the workstation 190 may generate and transmit control instructions to control the motion subsystem 210b, in remote system 104. In other cases, the input device memory 404i may, itself, store programs which can directly generate the control instructions from the encoder data. The I/O interface 412i can also receive control instructions from the workstation 190. For example, workstation 190 may receive position data from the remote motion system. The control instructions are generated by the workstation 190 to enable control of motors 444 to provide a haptic feedback effect to the user. In other cases, input device memory 404i may, itself, store programs which can directly generate the control instructions for motors 444 based on position data from the remote motion system.

Optionally, input device 180 may also include a communication interface, which may allow direct communication between the input device 180 and the remote system 104 (e.g., via network 110).

### Remote Robotic Control System

The following is a discussion of an example remote robotic control system, which may be used alone or in any combination or sub-combination with any other feature or features described herein (e.g., a remote ultrasound system, an input device, a local user control system, methods for operating a remote ultrasound system etc.).

As provided in greater detail herein, the remote control system may include a robotic arm. A distal end of the robotic arm may include an end effector, which can include a tool retention mechanism for retaining various tools required to perform ultrasound scanning (e.g., gel dispensers, ultrasound transducers, etc.). The robotic arm may be configured for up to six degrees of freedom of motion. The remote control system can also include one or more object detection cameras, which can be used to monitor the environment for any obstacles which may collide with the robotic arm.

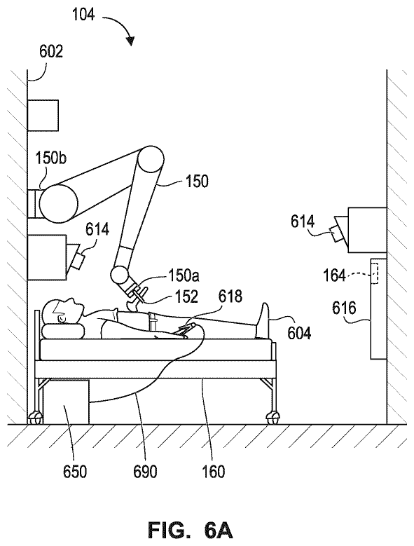

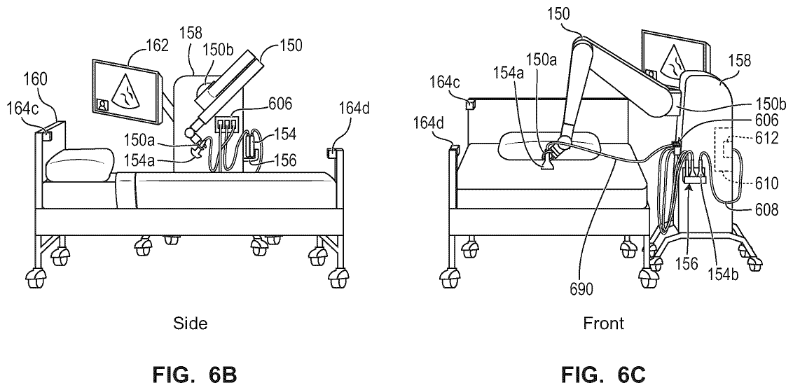

Reference is now made to FIGS. 6A-6C, which illustrates an example of a remote robotic control system 104.

As shown, the remote system 104 may include a motion subsystem 210b used for performing ultrasound scans. In the example illustrated, the motion subsystem 210b includes a robotic arm 150.

As further exemplified, the robotic arm 150 extends between a first arm end 150a and a distal second arm end 150b. First arm end 150a may be coupled to an end effector 152, which is used to retain and hold various tools used to conduct an ultrasound scan (e.g., ultrasound transducers, gel dispensers, etc.). Second arm end 150b may be mounted to a mounting surface, such as to support the weight of the robot arm 150. The mounting surface may include, for example, a wall 602 (FIG. 6A), e.g., a side-wall or ceiling in a scanning room. The mounting surface may also include a movable robot body 158 (FIGS. 6B, 6C). To this end, robot body 158 may allow the robotic arm 150 to be more easily transported between different environments.

As shown, the robotic arm 150 may be disposed such that at least the end effector 152 engages the patient 604. Where the patient is lying in the supine position (e.g., on bed frame 160)—robotic arm 150 may be positioned vertically above the patient. In other cases, the patient can be disposed in a standing position, in which case the robotic arm 150 may be positioned, for example, forward of the patient.

As provided herein, the robotic arm 150 may be adapted for several degrees of freedom of motion. The robotic arm 150—analogous to the input device 180—may be configured for up to six degrees of freedom of motion. This, in turn, enables the robotic arm 150 to vary the end effector's 152 spatial position (e.g., X, Y, Z cartesian position), as well as orientational position.

In particular, varying the end effector's spatial position allows the end effector 152 to access different positions over the patient's body, as well as more broadly, accessing different spatial positions in the remote environment (e.g., to access a tool holder for tool exchanges). Further, the ability to configure the end effector 152 in different orientational positions can further assist in using specifics tools. For example, as discussed, the ability to rotate, rock or roll an ultrasound transducer-attached to the end effector 152—can enable capturing more enhanced sonographic images.

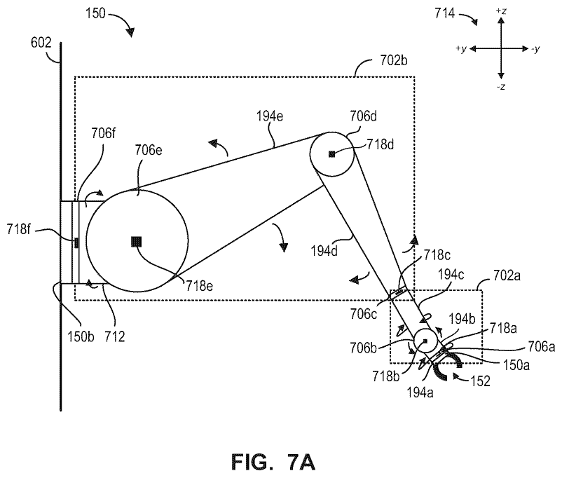

Reference is now briefly made to FIG. 7A, which shows a schematic illustration of an example robotic arm 150.

As shown, analogous to the input device 180, robotic arm 150 may include a plurality of arm segments 194a-194b which are rotatably connected via rotatable joints 706a-706f.

To enable six degrees of freedom of motion, the robotic arm 150 may include: (i) a first portion 702a for orientational position control of the end effector 152 (i.e., an orientation control portion 702a), and (ii) and a second portion 702b, for spatial position control of the end effector 152 (i.e., a spatial-position control portion 702b).

Analogous to the input device 180, orientational control portion 702a may include, (i) a forward “wrist” arm segment 194a for yaw or rotational control; (ii) a mid-arm rotation segment 194b for pitch or roll control; and (iii) a rear arm rotation segment 194c for roll control. The end effector 152 may be attached to the forward wrist arm segment 194a.

As shown, rotatable connection 706a rotatably connects the wrist arm segment 194a to the mid-arm segment 194b. Further, rotatable connection 706b rotatably connects the mid-arm segment 194b to the rear arm segment 194c.

It will be appreciated that arm segments 194a-194c may have a similar mode of operation as corresponding orientational-control arm segments 414a-414c in the input device 180, such as to also provide up to three degrees of freedom of motion.

Spatial-position control portion 702b, of the robotic arm 150, may also include, (i) a forward translational arm segment 194d; (ii) a rear translation arm segment 194e; and (iii) the rotating support member 712.

Arm segments 194d-194e, and rotating member 712, may have also have a similar mode of operation to corresponding arm segments 414d-414e and rotating member 412, respectively, in the input device 180. For example, forward arm segment 194d enables forward or backward translation of end effector 152 (i.e., moving the end effector 152 along the y-axis defined in a remote Cartesian reference frame 714). Rear translation arm segment 194e enables vertical upward and downward translation of the end effector 152 (i.e., z-axis translation in the remote reference frame 714). Rotating member 714 may allow “swinging” of the end effector 152 in the left and right directions (i.e., x-axis translation as defined in reference frame 714).

While not shown, a plurality of internal motor and shaft assemblies may control rotation of each of the rotating joints 706a-706f. The motors may be controllable by a controller or processor (e.g., processor 202b in FIG. 2) of the remote system 104 to achieve a desired positional configuration for the end effector 152.

Encoders (e.g., rotary encoders) 718a-718e may be connected to an end of each shaft, in each motor and shaft assembly. Encoders may generate encoder data, which may be used to determine the angular position of each joint 706a-706f. In turn, this data can be used to monitor and track the spatial and orientation position of the end effector 152. In other cases, any other position tracking sensors can be used for tracking position motion of the robotic arm 150. For example, an inertial measurement unit (IMU) can be mounted to end effector 152. The IMU may measure the pose (position and orientation) of the end effector 152 directly.

As provided herein, the robotic arm 150 may be operatable in various modes, including: (i) a non-autonomous, guided mode; (ii) a semi-autonomous, semi-guided mode; and/or (iii) a fully autonomous, non-guided mode.

As explained previously, in the guided mode, the robotic arm 150 may be fully controllable by the input device 180. That is, the robotic arm 150 may adjust the position of the end effector 152 (e.g., spatial and orientation position) based on control instructions received from the input device 180. In the semi-autonomous mode, the robotic arm 150 may be partially controllable by the input device 180. For example, the robotic arm 150 may be controlled in some cases by the input device 180, but may be autonomously operated in other cases. For example, the path motion of the robotic arm 150 may be automatically controlled to avoid detected obstacles. In a fully autonomous mode, the robotic arm 150 is otherwise fully autonomously controlled by a controller of the remote system 104.

FIG. 7B shows another example design configuration for the robotic arm 150.

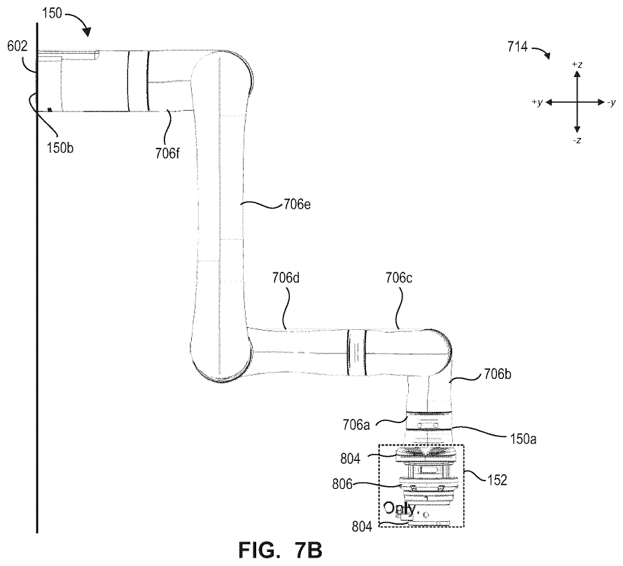

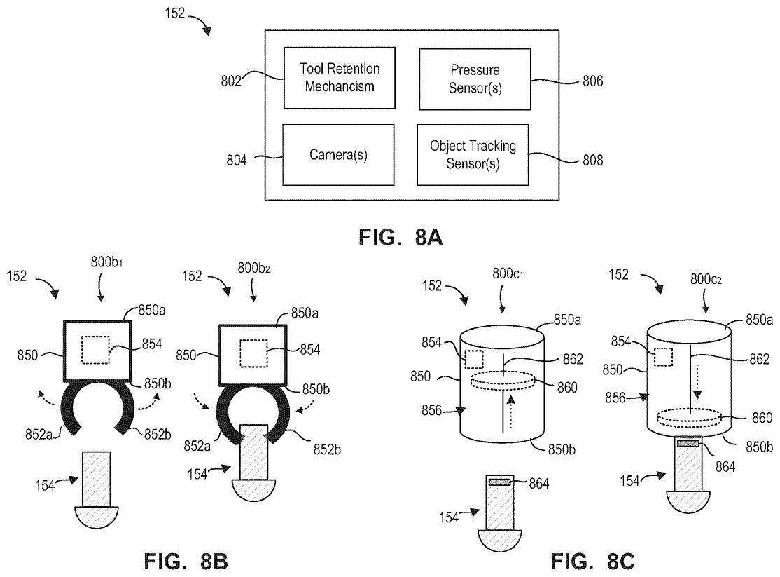

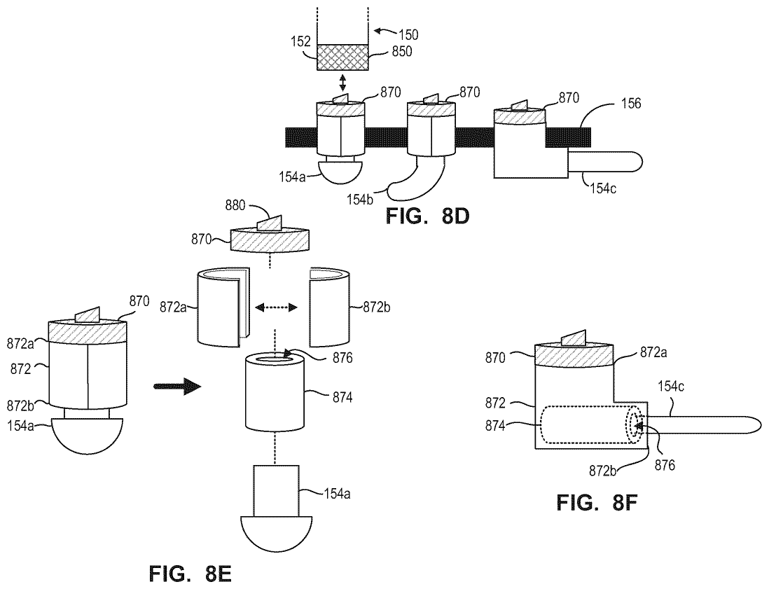

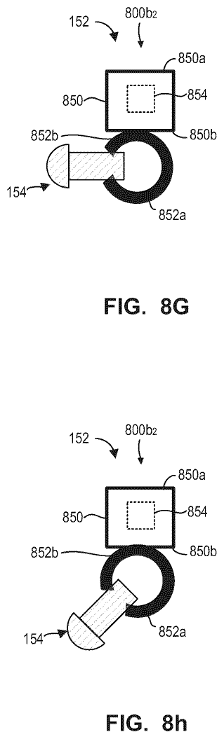

Referring now to FIG. 8A, which shows a simplified hardware block diagram of an example end effector 152, of the robotic arm 150.

As shown, the end effector 152 can include multiple sub-components, including a tool retention mechanism 802, and one or more camera(s) 804, pressure sensor(s) 806 and/or object detection sensor(s) 808.

Tool retention mechanism 802 can be used to retain and exchange various tools for use in ultrasound scanning (e.g., gel dispensers, ultrasound transducers, etc.). It will be understood that the tool retention mechanism 802 may have one of a number of possible configurations. FIGS. 8B-8F illustrate example configurations for the tool retention mechanism. In particular, FIG. 8B illustrates an example configuration using mechanical grippers, FIG. 8C illustrates an example configuration using permanent magnets and FIGS. 8D-8F illustrate an example configuration using mechanically coupling mechanisms.

Referring first to FIG. 8B, the end effector 512 can include a body portion 850 extending between opposite first and seconds ends 850a, 850b. First end 850a may be coupled to the robotic arm 150. Second end 850b may include mechanical grippers 852a, 852b for retaining various tools 154. As shown, grippers 852a, 852b may expand outwardly to disengage or release a tool (see 800b1), and may contract inwardly to engage or retain the tool (see 800b2). Once the tool is retained, the tool may be moved around and re-oriented by the robotic arm 150. In some example cases, the body portion 850 may house one or more servomotors 854, which can be used to control grippers 852.

While only two mechanical grippers 852 are illustrated in FIG. 8B, it will be understood that the end effector 152 may include any number of grippers.

In the example shown in FIG. 8B (and FIGS. 8C and 8D), the end effector 152 is configured to retain and manipulate a tool 154 in an orientation that is colinear with the direction along which the end effector 152 extends from the robotic arm 150.

Alternatively, the end effector 152 can be configured to retain a tool at a different position or angle. For example, the end effector 152 can be configured to retain the tool 154 at an offset position that is parallel to, but not colinear with, the direction along which the end effector 152 extends from the robotic arm 150.

Alternatively or in addition, the end effector 152 can be configured to retain a tool 154 at about a 90 degree or right angle as shown in the example of FIG. 8G. When the end effector 152 retains the tool 154 in a colinear orientation, this can introduce singularities into the determination of the motion path for the end effector 152. Adjusting the angle at which the tool 154 is retained can reduce these singularities.

As another example, the end effector 152 can be configured to retain a tool 154 at an angle between 0 and 90 degrees, e.g. about 45 degrees as shown in the example of FIG. 8H. This can reduce the singularities in the motion path determination while providing a more natural tool retention and manipulation position that can reduce inadvertent collisions with patients and other objects.

FIG. 8C illustrates another example configuration for the end effector 152. In the example shown, a permanent magnet 860 is disposed inside a hollow interior 856 of the end effector's body 850. Permanent magnet 860 is translatable between an unengaged position (800c1) and an engaged position (800c2). In the engaged position (800c2), the magnet 860 can be translated towards the second body end 850b. In this position, the permanent magnet 860 may attract a corresponding magnet 864 located inside, or on top, of a tool 154. Magnetic attraction between magnets 860, 864 allows the end effector 152 to retain the tool 154.

In the unengaged position (800c1), permanent magnet 860 can be translated away from the second house end 850b, and towards the first house end 850a. In translating towards the first end 850a, the magnetic attraction force is weakened between magnets 860, 864. This, in turn, allows the end effector 152 to disengage and release the tool 154 (e.g., to switch to a new tool).

In the illustrated example, the permanent magnet 862 may translate along an axial rail 862, as between the engaged and unengaged positions. One or more servomotors 854 can be used to translate the permanent magnet 862 along axial rail 862. In other examples, other translation mechanism can be used to translate magnet 862 between the engaged and unengaged positions.

Alternatively, rather than using a permanent magnet, an electromagnet may be used. The electromagnet may be disposed proximal the second end 850b of the end effector body 850. The electromagnet may be transitioned between an activated and an un-activated state. In the activated state, current is applied to the magnet to activate the magnet, and allow the magnet to engage magnet 864 associated with tool 154. In the un-activated state, the applied current is reduced or otherwise removed, thereby releasing the tool 154 from engagement with the end effector 152. The current may be applied using, for example, a power source located in the remote system 104.

FIG. 8D illustrates still another example configuration for the end effector 152. In this example configuration, the end effector 152 includes a mechanical connector. The mechanical connector is used to mate with a reciprocal connector associated with a tool 154.

In more detail, the end effector 152 may include a female connector which engages with a male connector 870 associated with tool 154. Any suitable mating engagement can be used as between the two connectors. For example, the tool's male connector 870 may include a protruding member 880. Protruding member 880 may be slidably receivable inside of a slot located in the end effector's female connector. In this manner, the end effector 152 can quickly engage and disengage various tools 154 located in the tool holder area 156.

In other cases, the female connector may be associated with the tool 154, while the male connector may be associated with the end effector 152.

As shown in FIG. 8E, each tool 154 may be retained within a housing casing 872. The housing casing 872 may have any suitable design. In the illustrated example, the housing casing 872 is formed of two casing portions 872a, 872b, which connect to clamp around at least a portion of the tool 154. Housing casing 872 may further extend between a first end 872a and a second end 872b. First end 870a may couple to the connector 870, while second end 870b may include an opening through which the tool 154 protrudes. In the case of a transvaginal probe 154c, casing 872 can have a right-angle design, to facilitate insertion of the probe into a patient's vagina (FIG. 8F).

In some example cases, housing casing 872 may also retain a flexible sock 874. The flexible sock 874 can include a hole 876, which can receive at least a portion of the tool 154 within the casing 872. The flexible sock 874 can provide a degree of freedom of movement between the tool 154 and the casing 872. For example, this may allow the transvaginal probe 154c to have some flexibility of movement when inserted, and navigated inside the patient's vagina. In turn, this prevents otherwise injuring the patient, as may occur where the probe 154c is rigidly affixed to the housing casing 872.

Referring back to FIG. 8A, the end effector 152 may also include one or more of cameras 805, pressure sensors 806 and/or object tracking sensors 808.

Camera(s) 804 may positioned on the end effector 152 to assist in capturing various images and video feeds (e.g., live video feeds) (see e.g., downward facing camera 804 in FIG. 7B). These images and video feeds may be transmitted back to the local control system 102, and may be displayed to the local user operator (e.g., on computer display 190b, in FIG. 2). This may allow the operator to more closely track the position of the end effector 152 relative to the patient. To this end, the camera(s) 804 may form part of the remote system's A/V subsystem 206b (FIG. 2).

Pressure sensor(s) 806 can be used to monitor the pressure applied by the end effector 152 to the patient. For example, the pressure sensor(s) 806 can monitor the applied pressure to determine whether the pressure is within an acceptable range. In turn, this can ensure that the robotic arm 150 is not otherwise applying excessive force to the patient, which may otherwise injure the patient.

In some example cases, if the recorded pressure data is above a pre-determined threshold, the remote system 102 may either disable the robotic arm 150, or otherwise automatically adjust the robotic arm 150 to minimize applied pressure. In some cases, “disabling” the robotic arm 150 may involve automatically moving the end effector 152 to a pre-determined spatial position, that is spaced away from the patient.

In some example cases, pressure data generated by pressure sensor(s) 806 may be transmitted to the local system 102 to allow a local operator to monitor applied pressure during the scanning process.

Example pressure sensor(s) 806 that can be incorporated into the end effector 152 include various types of load cells, including pneumatic load cells, capacitive load cells, strain gauge load cells and/or hydraulic load cells. In some cases, multiple pressure sensors 806 may be provided on the end effector 152 to monitor applied force, or pressure, from multiple directions.

Object tracking sensor(s) 808 can include, for example, various object tracking cameras, including time-of-flight (ToF) sensors. The object tracking sensor(s) 808 can be used to automatically detect obstacles in the end effector's path. In the semi-guided and autonomous modes, motion data-generated by sensor(s) 808—can be used to automatically control the robotic arm 150 to avoid detected obstacle. Sensor(s) 808 may also, more broadly, be used to detect position and movement of the patient to enable for precision tool localization relative to the patient, as well as adapting movement of the robotic arm 150 relative to the patient's motion.

Referring back to FIGS. 6B and 6C, the various tools required for performing ultrasound scanning may be retained in a tool holder area 156. For example, the tool holder 156 may retain various types of ultrasound transducers, as well as gel dispensers. The tool holder 156 may be, for example, installed on a lateral side of the robot body 158.

To this end, when it is desired to exchange tools, the remote system 104 may translate the robotic arm 150 and end effector 152 to the tool holder area 156. The end effector 152 may then disengage (or drop-off) a currently retained tool, and engage (or pick-up) a new tool.

Optionally, the remote control system 104 may have advanced knowledge of the exact position location of the tool holder area 156 within the remote reference environment (e.g., X, Y, Z positional location). This information can be stored, for example, in memory 204b of the remote control system 104.

Accordingly, each time it is desired to exchange a tool, the remote system 104 may automatically control the robotic arm 150 to translate and align the end effector 152 with the known position of the tool holder area 152. In the guided or semi-guided modes, this may occur in response to receiving a user input command—from the local system 102—to exchange tools. In the fully autonomous mode, this can occur automatically in response to the system determining a new tool is required.

In some other cases, the remote control system 104 may also have knowledge of the exact location of each tool within the tool holder area 156. For example, each tool may be associated with a pre-defined coordinate position in the remote reference frame (e.g., X, Y, Z position), corresponding to its designated location in the tool holder area 156. Accordingly, the remote system 104 may reference this coordinate position when it is required to drop-off a tool, or otherwise pick-up a tool. In some cases, the control system memory 204b may store a table, or database, of tools and their associated coordinate positions in the remote reference frame.

It will be appreciated that, in various cases, tools 154 may not necessarily be aggregated into a single tool holder area 156. For example, it is possible that the tools are dispersed in different locations within the remote environment.

As further shown in FIGS. 6B and 6C, electronic tools 154 may be connected via data cables 690 to transmit, or receive data. For example, a data cable 690 may connect an ultrasound transducer 154a to a controller of the remote system 104. Controller may include, not only a processor 202b of the remote system 104 (FIG. 2), but also various drivers required to operate the ultrasound transducer. In the illustrated example, the controller may be housed within the robot body 158, and the data cables 690 may connect to input ports 606 disposed on the robot body 158. In other cases, the controller may be provided in an external housing 650 (FIG. 6A).

To this end, data cable 690 can be used to transmit commands to vary configuration parameters of the ultrasound transducer 154a. For example, these may include user commands received from the local system 102. These may also include commands automatically generated by remote system 104. Transducer 504a may also transmit sonographic images to the controller, via data cable 604.

In other cases, some of the electronic tools 154 may also be adapted for wireless communication.

In the case of gel dispensers 154b, the gel dispensers 154b may be connected via tubing 608 to a gel tank 610. The gel tank 610 can supply gel to the gel dispenser 154b through a controllable pump mechanism 612, i.e., controlled by the remote system controller. In other cases, the gel can be gravity fed from the gel tank 610.

In some cases, the remote system 104 may monitor the amount of gel consumed in the gel tank 610. For example, this may be performed via a level sensor positioned inside the gel tank 610. In other cases, the monitoring may be based on the known maximum fill volume of the gel tank 610, in conjunction with the known amount of previously dispensed gel. The amount of dispensed gel can be determined, for example, based on the period of time the pump 612 was activated in combination with the known flow rate of the pump 612. When it is determined that the quantity of remaining gel is below a pre-determined threshold, an alert can be generated to refill the gel tank 610.

Alternatively, a gel dispenser can be provided in the form of a disposable gel container (e.g. a plate or jar). Gel can be provided by the gel container and the end effector 152 can manipulate an ultrasound transducer probe to extract gel from the container (e.g. by dipping the probe into the container). The probe can then be manipulated to dispense the gel onto the patient. This may avoid challenges caused by gel become stuck or dried within a dispensing tube.

As shown in FIG. 6A, remote system 104 may also include one or more object tracking sensors 164, which are installed around the environment. Object tracking sensors 164 can include, for example, object tracking cameras (e.g., LiDAR or other time-of-flight sensors) to monitor the environment surrounding the robotic arm 150.

Analogous to object tracking sensor 808 in the end effector 152—object tracking sensors 164 can be used to detect obstacles in the remote environment, such as to prevent a collision between the obstacle and the robotic arm 150. For example, in response to detecting an obstacle, the remote system 104 may either stop movement of the robotic arm 150, and await further instructions from the local system 102. Otherwise, in the semi-guided and fully autonomous modes, the remote system 104 may automatically plan the robot's arm motion around the obstacle. Object tracking sensors 614 may also, more broadly, be used to detect position and movement of the patient to again enable for precision tool localization relative to the patient, as well as adapting movement of the robotic arm 150 relative to the patient's motion.

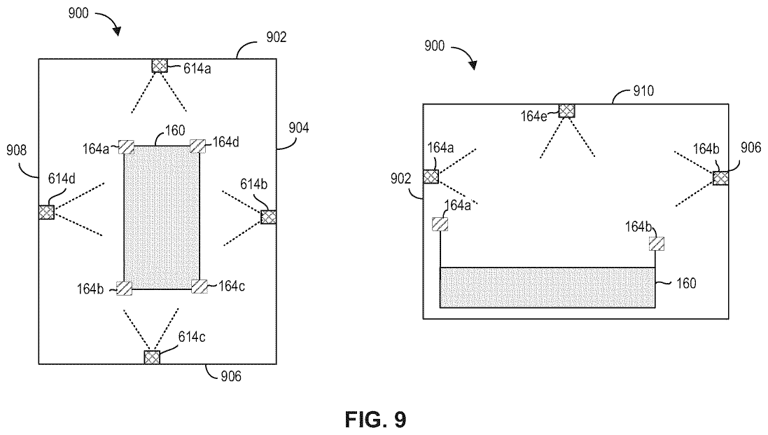

As shown in FIG. 9, the object tracking sensors 614 may be positioned in different configurations around the remote environment 900 to enable motion capture from different angles. For example, object tracking sensors 614a-614e may be positioned along different lateral sidewalls 902-908 of a scanning room 900, as well as on a ceiling 910.

The remote system 104 can also, more generally, include one or more cameras 164 for generating a video feed (e.g., a real-time or near real-time feed). The feed generated by the cameras 164 can be transmitted back to the local system 102. In particular, the camera feeds may enable a local user operator to monitor the patient environment, as well as observe and track the robot arm motion from multiple perspectives.

As shown in FIG. 9, cameras 164a-164d may also be positioned at different locations around the bed frame 160. Each camera 164 can therefore provide a different perspective angle of the patient, as well as of the robot arm 150. Cameras 164 may also be installed in other locations, such along one or more of the room's lateral sidewalls 902-908, or the ceiling 910. The cameras 164 may be configured to be remotely controlled (e.g. panned, tilted, zoomed) by the local control system 102 in response to operator input. This can allow the operator to adjust the view of the patient environment in response to changes in the environment, e.g. patients in different positions or of different sizes.

To this end, cameras 164a-164d may form part of the remote system's A/V subsystem 206b (FIG. 2). In some example cases, as best shown in FIG. 6A, the A/V subsystem 206b may also include a display device 616, which may display a video feed of the local operator to the patient. In this manner, the remote local operator may interact with the patient to provided guided instructions, while also allowing the patient to interact with the local operator. The A/V subsystem 206b may also include speakers and microphones to transmit and receive audio.

As best shown in FIG. 6A, the remote system 104 can also include a pain threshold monitor 618, which is analogous to the patient feedback device 216b (FIG. 2). Pain threshold monitor 618 can be activated by the patient when the robot system is applying excessive, or uncomfortable force to the patient. Once activated, the threshold monitor 618 may transmit an activation signal to the system processor 202b. In response, the remote system may either reduce the force applied by the robot arm 150 to the patient, or may otherwise entirely disengage the arm 150. In disengaging arm 150, the system may either move the arm 150 to a pre-determined default position away from the patient, or may otherwise stop the arm 150 from any further movement. In some cases, the system may also cut-off power to the arm 150 when the pain threshold monitor 618 is activated.

The pain threshold monitor 618 may have any suitable design. For example, the monitor 618 may include a clickable button. In some example cases, the button may be pressed to generate the activation signal. In other cases, the user may initially hold the button, and may then release the button to generate the activation signal.

Alternatively, the pain threshold monitor 618 can include a hand clamp-type mechanism, which may be either squeezed or released to generate the activation signal. In some cases, the clamp may incorporate a pressure sensor, which can monitor the pressure applied by the user.

The pain threshold monitor 618 may not necessarily generate a binary output (i.e., activated or inactivated). Rather, the monitor 618 can generate levels, graduation, or intensities of activated signals. For example, where the monitor 618 includes a hand clamp, the user may vary the degree of squeezing or releasing of the clamp to indicate different levels of experienced pain. The system may then adjust and accommodate for the intensity of the received activation signal by decreasing the applied force, by the robot arm 150, in linear or non-linear proportion.

### Methods for Operating a Remote Ultrasound System

The following is a discussion of example methods for operating a remote ultrasound system, which may be used alone or in any combination or sub-combination with any other feature or features described herein (e.g. a remote ultrasound system, an input device, a local user control system, a remote robotic control system etc.).

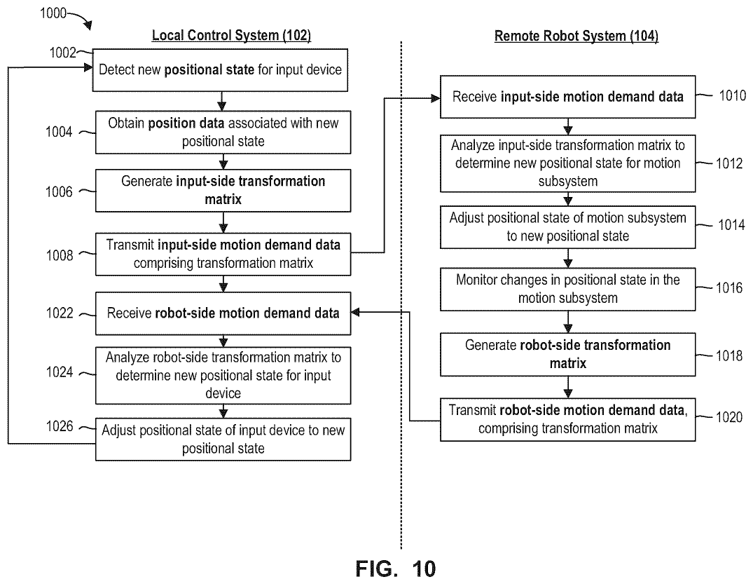

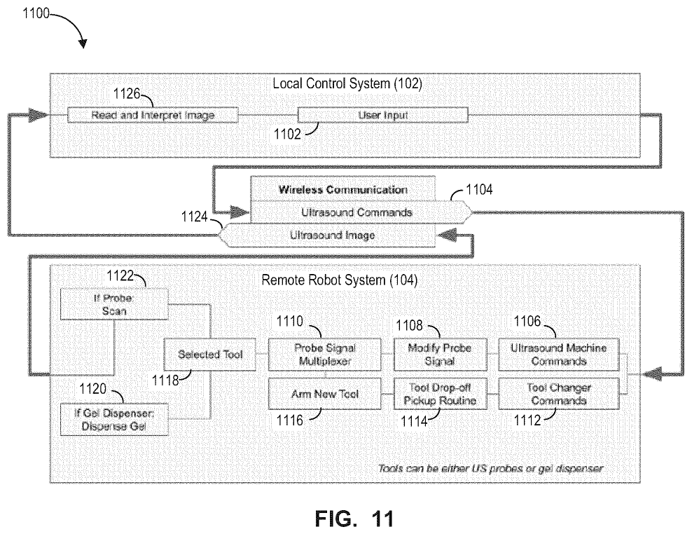

Reference is now made to FIG. 10, which shows a process flow for an example method 1000 for controlling the remote robot system 104 using the input device 180 of the local control system 102. Method 1000 may be performed, for example, when the remote robot system 104 is operated in either the non-autonomous, guided mode, or otherwise, the semi-autonomous, semi-guided mode.

As shown, at 1002, the local control system 102 may detect a new positional state for the input device 180. For example, where the input device 180 includes a user-controllable arm system 402 (i.e., FIGS. 4A-41), act 1002 may involve detecting spatial or rotational positional changes in the arm system 402. The positional changes may result, for example, from the local operator engaging and applying force to the joystick 450. To this end, detecting positional changes at act 1002 may involve detecting new sensor data generated by the input device's sensor subsystem 410i (FIG. 4I). In particular, this can include detecting new encoder data generated by encoders 440a-440f, which are coupled to the various rotating shafts in the input device 180, as previously described.

If a positional change is detected, at 1004, the local control system 102 can obtain position data associated with the input device's new positional state. For example, this may involve obtaining the encoder data, generated by each encoder 440a-440f, associated with each rotatable connection in the input device 180 (e.g., the rotatable connections between various arm segments 414a-414e, as well as the rotatable member 412). Optionally, at act 1004, the local control system 102 may perform initial filtering of the obtained encoder data to generated filtered encoder data (or more generally, filtered sensor data).

At 1006, the local control system 102 can process the new position data (e.g., encoder data) to solve a forward kinematics model. For the input device 180 of FIGS. 4A-41, the forward kinematics model may express a new position for the joystick 450 (e.g., new spatial and orientational position). An input-side transformation matrix may then be generated, which includes various parameters of the solved forward kinematics model.

At 1008, the local control system 102 may transmit, to the remote system 104, input-side motion demand data. The input-side demand data can include the determined input-side transformation matrix. As explained herein, the input-side motion demand data constitutes the control instructions for controlling the motion subsystem 210b (e.g., the robotic arm 150) in the remote environment.

At 1010, the remote robot system 104 can receive the input-side motion demand data, e.g., via network 110, from the local control system 102.

At 1012, the remote robot system 104 can analyze the input-side transformation matrix to determine a new positional state for the motion subsystem 210b. to this end, act 1012 can involve solving an inverse kinematics model based on the parameters included in the input-side transformation matrix. In example cases where the motion subsystem 210b includes a robotic arm 150, solving the inverse kinematics model can allow determining new joint angles for each of the robot arm's joints 706a-706f.

At 1014, the remote robot system 104 can adjust the motion subsystem 210b to the new positional state. That is, the remote robot system 104 can manipulate the motion subsystem 210b to generate a new position for the end effector 152. In example cases where the motion subsystem 210b includes a robotic arm 150, act 1014 may involve controlling the arm's internal motors—which are connected to each of the robot arm's joints 706a-706f—to achieve desired target joint angles. A proportional-integral-derivative (PID) loop can be used to achieve the new position for the end effector in the motion subsystem.

At 1016, the remote robot system 104 can monitor for a change in the positional state of the motion subsystem 210b (e.g., change in the spatial or orientational position state). That is, the remote robot system 104 can monitor if there has been a change to the position state of the system's end effector 152 from the last position generated at act 1014. In various example cases, the change in positional state may result where an external force is applied to the motion subsystem, such as where the motion subsystem 210b collides with an obstacle (e.g., the patient's body, or an external object).

In at least some example cases, where the motion subsystem 210b includes a robotic arm 150—act 1016 can be performed by monitoring data generated by sensors in the robotic arm 150. For example, this can involve monitoring encoder data generated by each of the robot arm's encoders 718a-718e, which are coupled to the various arm joints 706a-706f (FIG. 7).

At 1018, the remote robot system 104 may generate a robot-side transformation matrix. The robot-side transformation matrix may be generated by solving a forward kinematics model based on, for example, encoder data generated by the robot arm's encoders 718a-718e. In at least some cases, the sensor data (e.g., the encoder data) may be initially pre-filtered to generate filtered sensor data. The filtered sensor data may then be used to solve the forward kinematics models to generate the robot-side transformation matrix.

At 1020, the remote robot system 104 may transmit, to the local control system 102, robot-side motion demand data that includes the robot-side transformation matrix.

At 1022, the local control system 102 may receive the robot-side motion demand data, e.g., via network 110.

At 1024, the local control system 102 can analyze the robot-side transformation matrix, included in the received robot-side motion demand data. Based on the analysis, the local control system 102 can determine a new positional state for the input device 180 (e.g., new spatial and orientational state). For example, where the input device 180 includes the user-controllable arm system 402, act 1024 may involve determining a new positional state for the joystick 450. This may be determined, for example, by solving an inverse kinematics model based on the received robot-side transformation matrix. Solving the inverse kinematics model may allow for determining new angles for each rotatable connection in the input device 180.

At 1026, the input device 180 may be controlled to adjust its positional state to the determined new positional state. For example, this may involve controlling the motors 444a-444e, in the input device's motor subsystem 406i, to adjust the angle of each rotatable connection in the input device 180. In turn, this can adjust the spatial and/or orientational position of the joy stick 450 in the input device 180. For example, a PID loop can be used to achieve the new positional state.

Here, it will be appreciated that acts 1016-1026 provide for the haptic feedback effect in the input device 180, as the input device 180 is controlled to mirror external forces that may be applied to the remote motion subsystem at act 1016.

Subsequent to act 1026, method 1000 may return back to act 1002 to again detect a new positional state for the input device 180, i.e., resulting from interaction of the local user operator with the input device 180.

Optionally, prior to performing act 1002—the local control system 102 and remote robot system 104 may perform an initialization protocol. The initialization protocol may involve the remote robot system 104 generating and transmitting an initial robot-side transformation matrix to the local control system 102. The initial robot-side transformation matrix may reflect the motion subsystem's initial positional state. The local control system 102 may also generate its own input-side transformation matrix, corresponding to the input device's initial positional state.

The local control system 102 may then analyze the robot-side transformation matrix to determine an offset of its origin frame to the robots origin frame based on the difference between the two transformation matrices. This may be performed to account for the significantly smaller work volume of the input device 180 as compared the remote robot system's workspace. The systems 102, 104 may then use this dynamically offset representation of the input device's work volume in the robot's workspace to obtain one-to-one (or a scaled value) position matching of the input device position to the position of the end-effector in the remote robot workspace (also known as traversing the robot workspace).

Reference is now made to FIG. 11, which shows an example control data flow 1100 between the local control system 102 and the remote robot system 104 for controlling various configuration parameters of the remote robot system 104.

As shown, at 1102, a local user operator may input user commands 1102 into the local control system 102. For example, this may include commands to vary or change a tool being currently used by the remote system 104. The input commands can also include commands to adjust various ultrasound configuration parameters of ultrasound transducers being used by the remote system 104. The user inputs may be received, for example, at the input interface 190a of the local control system 102.

At 1104, the user input commands are transmitted to the remote robot system 104, e.g., via the network 110.

At 1106-1122, the remote robot system 104 may receive and execute the received commands. For example, at acts 1106-1110, the remote robot system 104 may execute commands to adjust ultrasound parameters for a currently-deployed ultrasound transducer. This may involve, for example, receiving the ultrasound machine command (1106), and in response, either modifying a probe signal associated with the ultrasound transducer based on the received command (1108) and/or controlling the ultrasound probe signal multiplexer (1110).

In other cases, at acts 1112-1116, the remote robot system 104 can receive and execute a command to pick-up a new tool, or otherwise exchange a currently-held tool for a new tool. In particular, the remote robot system 104 can receive the tool change command (1112), and in response, the remote robot system 104 may control the motion subsystem 210b to either drop-off a currently-held tool and pick-up the new target tool (1114), or otherwise simply pick-up the new target tool (1116). The target tool may be, for example, an ultrasound transducer or a gel dispenser.

At act 1118, the remote system 104 may use a currently-held (or currently-selected) tool. For example, if the tool is gel dispenser, the remote robot system 104 can control the gel dispenser to deposit gel on a desired area of the patient's body, i.e., where a scan is to be performed. In some example cases, act 1118 may involve controlling the gel dispenser pump 612 to deposit gel (FIG. 6C).

In other cases, at 1122, if the tool is an ultrasound transducer, then the remote system 104 can use the ultrasound transducer to perform an ultrasound scan. To this end, the transducer can be configured according the configuration parameters selected at acts 1108-1110. Once the scan is completed, the remote system 104 may then generate and transmit an ultrasound image to the local system, i.e., via network 110 (1124). The local system 102 may receive the ultrasound image (1126), which can then be read and interpreted by the local system operator (e.g., the sonographer). Data flow 1100 may then return to act 1102 to iterate until the local user operator has obtained the desired ultrasound images.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

1. A system for remote ultrasonography, comprising:
a local control system comprising an input device and at least one local processor coupled to the input device;
a remote robotic ultrasound system comprising a motion subsystem and at least one remote processor coupled to the motion subsystem,
wherein the at least one local processor, of the local control system, is operable to:
detect a new positional state for the input device;
obtain positional data corresponding to the new positional state;
generate, based on the positional data, input-side motion demand data; and
transmit the input-side motion demand data to the remote robotic ultrasound system,
wherein the at least one remote processor, of the remote robotic ultrasound system, is operable to:
receive the input-side motion demand data; and
adjust a positional state of the motion subsystem, based on the input-side motion demand data.

2. The system of claim 1, wherein the at least one remote processor, of the remote robotic ultrasound system, is further operable to:
monitor for changes in the positional state of the motion subsystem; and
in response to detecting a change in the positional state, generate robot-side motion demand data corresponding to a new position state of the motion subsystem; and
transmit the robot-side motion demand data to the local control system.

3. The system of claim 2, wherein the at least one local processor, of the local control system, is further operable to:
receive the robot-side motion demand data; and
adjust a positional state of the input device, based on the robot-side motion demand data, so as to generate a haptic feedback effect at the input device.

4. The system of claim 1, wherein the input device further comprises a sensor subsystem, and detecting a new positional state for the input device comprises receiving new sensor data from the sensor subsystem.

5. (canceled)

6. (canceled)

7. The system of claim 1, wherein generating the input-side motion demand data comprises determining an input-side transformation matrix, based on the positional data, wherein determining the input-side transformation matrix comprises solving a forward kinematics model.

8. The system of claim 1, wherein adjusting a positional state of the motion subsystem comprises analyzing the input-side transformation matrix by solving an inverse kinematics model.

9. The system of claim 2, wherein generating robot-side motion demand data comprises determining a robot side transformation matrix, based on the new positional state, wherein determining the robot-side comprises solving a forward kinematics model.

10. The system of claim 1, wherein the input device comprises a movable arm system.

11. The system of claim 1, wherein the motion subsystem comprises a robotic arm.

12. The system of claim 1, wherein the motion subsystem comprises a tool-retaining end effector for retaining a tool.

13. (canceled)

14. The system of claim 12, wherein the tool comprises one or more of an ultrasound transducer, gel dispenser and a transvaginal probe.

15. (canceled)

16. (canceled)

17. The system of claim 1, wherein the local control system further comprises an input interface, and at least one local processor of the local control system is further operable to:
receive one or more user inputs from the input interface; and
transmit the one or more user inputs to the remote robotic ultrasound system.

18. (canceled)

19. (canceled)

20. The system of claim 1, wherein the remote robotic ultrasound system further comprises a pain threshold monitor coupled to the at least one remote processor of the remote robotic ultrasound system, and wherein the at least one remote processor of the remote robotic ultrasound system is further operable to:
monitor for an activation signal from the pain threshold monitor; and
in response to detecting the activation signal, one of disabling the motion subsystem and reducing an applied force of the motion subsystem on a patient.

21. A method for remote ultrasonography, comprising:
detecting a new positional state for an input device of a local control system;
obtaining positional data corresponding to the new positional state;
generating, based on the positional data, input-side motion demand data; and
transmitting, from the local control system, the input-side motion demand data to a remote robot system,
receiving, at the remote robot system, the input-side motion demand data; and
adjusting a positional state of a motion subsystem, of the remote robot system, based on the input-side motion demand data.

22. The method of claim 21, further comprising:
monitoring, at the remote robot system, for changes in the positional state of the motion subsystem; and
in response to detecting a change in the positional state, generating robot-side motion demand data corresponding to a new position state of the motion subsystem; and
transmitting the robot-side motion demand data to the local control system.

23. The method of claim 22, further comprising:
receiving, at the local control system, the robot-side motion demand data; and
adjusting, at the local control system, a positional state of the input device, based on the robot-side motion demand data, so as to generate a haptic feedback effect at the input device.

24. The method of claim 1, wherein the input device comprises a sensor subsystem, and detecting a new positional state for the input device comprises receiving new sensor data from the sensor subsystem.

25. (canceled)

26. (canceled)

27. The method of claim 21, wherein generating the input-side motion demand data comprises determining an input-side transformation matrix, based on the positional data, wherein determining the input-side transformation matrix comprises solving a forward kinematics model.

28. (canceled)

29. (canceled)

30. (canceled)

31. (canceled)

32. (canceled)

33. (canceled)

34. (canceled)

35. (canceled)

36. (canceled)

37. The method of claim 21, wherein the local control system further comprises an input interface, and the method further comprises:
receiving, at the local control system, one or more user inputs from the input interface; and
transmitting, from the local control system, the one or more user inputs to the remote robotic ultrasound system.

38. (canceled)

39. (canceled)

40. (canceled)

41. An input device comprising:
a mounting structure;
a user-controllable arm system, comprising: (i) a mechanical arm extending between a first arm end and a distal second arm end, and (ii) a rotatable member rotatably coupled to the mounting structure, wherein the mechanical arm is secured to the rotatable member at the second arm end;
a motor subsystem comprising a plurality of motors for controlling a positional state of the arm system;
a sensor subsystem for monitoring the positional state of the arm system;
at least one processor coupled to each of the motor subsystem and the sensor subsystem.

42.-65. (canceled)
